(19) United States
(12) Patent Application Publication
(10) Pub. No.: US 2026/0210765 A1
(43) Pub. Date: Jul. 23, 2026

(54) METHOD FOR DETERMINING AT LEAST ONE COLORIMETRIC PARAMETER CHARACTERIZING A COSMETIC COMPOSITION, ASSOCIATED ELECTRONIC DETERMINATION DEVICE AND COMPUTER PROGRAM PRODUCT

(71) Applicant: L'OREAL, Paris (FR)

(72) Inventors: Julien BEUTIER, Clichy-la-Garenne (FR); Telma DA SILVA, Clichy-la-Garenne (FR); Vincent AURIAU, Clichy-la-Garenne (FR)

(73) Assignee: L'OREAL, Paris (FR)

(21) Appl. No.: 19/139,915

(22) PCT Filed: Dec. 28, 2023
(86) PCT No.: PCT/EP2023/087878

§ 371 Date: Jun. 17, 2025

(30) Foreign Application Priority Data
Dec. 30, 2022 (FR) 2214698
Dec. 30, 2022 (FR) 2214700
Dec. 30, 2022 (FR) 2214702

(51) Int. Cl. G01J 3/46; A45D 44/00; G16C 20/30; G16C 20/70

(52) U.S. Cl. CPC G01J 3/46; A45D 44/005; G16C 20/30; G16C 20/70

(57) ABSTRACT

The present invention relates to a method for determining at least one colorimetric parameter characterizing a cosmetic composition (11) for coloring hairs (12), in particular hair. The method comprises a learning phase comprising receiving a set of viability constraint(s), acquiring a set of training data and training an artificial intelligence model using the set of training data to obtain a trained model.

The method further comprises a processing phase including obtaining a data item under test and the applying the trained model to the data item under test to determine the at least one associated colorimetric parameter, the data item under test complying with the set of viability constraint(s).

A first invention relates to a method for determining at least one colorimetric parameter. The present invention also relates to an associated computer program product and electronic determination device.

The present first invention relates to the field of cosmetic products, preferably cosmetic compositions for coloring hairs, in particular hair.

More generally, a “cosmetic product” is a product as defined in Regulation EC No. 1223/2009 of the European Parliament and of the Council of Nov. 30, 2009, relating to cosmetic products.

An objective of the cosmetic industry is to improve the experience of its consumers. There is in particular a strong trend to propose products that are increasingly adapted to the needs and characteristics specific to the user. This trend is generally called “customization”.

Customizing cosmetic products and services can concern any part of the human body but is of particular interest for exposed body parts, such as the face (makeup or care products), and the hair or beard where applicable (coloring products for example).

In the field of cosmetic compositions for coloring hair(s), in particular hair, novel cosmetic compositions are regularly developed to adapt to user demand.

These novel cosmetic compositions are aimed, in a first case, at achieving a novel color or a novel visual effect of the hairs on which they are applied. In a second case, these novel cosmetic compositions are aimed at achieving a previously known color but comprise ingredients not previously used for this purpose. This second case is of particular interest when the procurement of certain ingredients becomes difficult, is accompanied by high costs, or poses a risk for the environment.

However, obtaining novel cosmetic compositions results from a lengthy and complex process.

Firstly, expert chemists develop cosmetic composition prototypes. These prototypes result from the experience of the expert chemists and are governed by certain chemical principles.

Then, these prototypes are tested individually in a laboratory to check that the visual effect obtained meets the expert chemists' expectations.

In some cases and unpredictably, the visual effect associated with a newly developed prototype does not meet expectations. It is therefore not possible to capitalize on the work of the expert chemists who developed these prototypes.

Therefore, there is a need for tools making it possible to simplify the development of a novel cosmetic composition.

The first present invention proposes to overcome this problem by using artificial intelligence to assist with the development of novel cosmetic compositions.

The first present invention relates to a method for determining at least one colorimetric parameter characterizing a cosmetic composition for coloring hairs, in particular hair, the method being implemented by an electronic determination device and comprising a training phase including the following steps:
receiving a set of viability constraint(s) of cosmetic composition(s),
acquiring a set of training data, each training data item being specific to a cosmetic composition complying with the set of viability constraint(s) received, each training data item comprising:
   a set of magnitudes representing the quantity of each ingredient of the cosmetic composition, and
   a value of the at least one colorimetric parameter associated with the cosmetic composition, and
training an artificial intelligence model based on the set of training data, to obtain a trained model,
   the method further comprising a processing phase including the following steps:
obtaining a test data item relating to a cosmetic composition under test, the cosmetic composition under test complying with the set of viability constraint(s) received during the training phase,
the test data item comprising a set of magnitudes representing the quantity of each ingredient of the cosmetic composition under test,
applying, to the test data item obtained, the trained model to determine the value of the at least one colorimetric parameter of the cosmetic composition under test.

Thanks to the artificial intelligence model, it is possible to determine quickly and simply, the at least one colorimetric parameter associated with each cosmetic composition under test.

Furthermore, the fact that each cosmetic composition complies with the set of constraint(s) makes it possible to benefit from the expert chemists' experience and avoid unnecessarily testing cosmetic compositions which would not be viable.

According to particular embodiments, the determination method according to the first invention comprises one or several of the following characteristics, taken in isolation or in any technically possible combination:
each ingredient of the test data item relating to the cosmetic composition under test is chosen from a predefined list of ingredients;
during the obtaining step of the processing phase, several test data items are obtained, each test data item being specific to a respective cosmetic composition under test,
during the application step of the processing phase, the trained model is applied to each test data item obtained to determine a value of the at least one colorimetric parameter associated with said test data item, the obtaining step of the processing phase comprising:
   receiving a number N corresponding to a number of cosmetic compositions under test, the number N being greater than or equal to two,
   generating N test data items,
each test data item relating to a cosmetic composition of which the ingredients are chosen from the predefined list of ingredients and such that the cosmetic composition associated with each test data item is within a reduced viable cosmetic composition space,
the reduced viable cosmetic composition space only comprising cosmetic compositions complying with the set of viability constraint(s), and the N test data items being representative of said reduced viable cosmetic composition space;
during the obtaining step of the processing phase, generating N test data items of the obtaining step comprises computing magnitudes of the N test data items such that, for each test data item, the distance between said test data item and the other test data items is maximum,
each magnitude of each test data item representing the quantity of an ingredient in the corresponding cosmetic composition under test;
the predefined list of ingredients comprises one or more ingredients of a first type, and one or more ingredients of a second type,
the or each ingredient of the first type being a base, and
the or each ingredient of the second type being a coupler;
the set of cosmetic composition viability constraint(s) comprises one or more of the following constraints:
   a ratio between a quantity of ingredient of the first type and a quantity of ingredient of the second type is between a first threshold and a second threshold,
   a total quantity of the cosmetic composition is less than a third threshold,
   a quantity of each ingredient in the cosmetic composition is less than a fourth threshold,
   a number of ingredients in the cosmetic composition is less than a fifth threshold;
the at least one colorimetric parameter comprises:
   a triplet of values characterizing a hair color after applying the cosmetic composition, or
   a color fade value of the cosmetic composition after washing the hairs, or
   a selectivity value characterizing a difference in color between a root and a tip of the hairs;
during the acquisition step of the training phase, the set of training data is, at least in part, acquired via a sensor capable of measuring the at least one colorimetric parameter;
the processing phase further comprises a step of sending the or each colorimetric parameter and the associated test data item, to a display screen, with a view to displaying on the display screen, a rendering for the or each cosmetic composition under test, the or each rendering being representative of the application, to the hairs, of the cosmetic composition under test; and
the processing phase further comprises a step of producing at least one sample of cosmetic composition under test, with a view to applying it on hairs to verify the value of the at least one colorimetric parameter.

The present first invention also relates to a computer program product on which a computer program comprising program instructions is stored, the computer program being loaded onto a data processing unit and implementing such a method when the computer program is implemented on the data processing unit.

The present first invention also relates to an electronic device for determining at least one colorimetric parameter characterizing a cosmetic composition for coloring hairs, in particular hair, the electronic determination device being capable of implementing such a determination method.

The present first invention also relates to a readable information medium on which a computer program product comprising program instructions is stored, the computer program being loaded onto a data processing unit and implementing such a determination method when the computer program is implemented on the data processing unit.

Other features and advantages of the first invention will appear when reading the following description of embodiments of the invention, provided solely as an example and with reference to the drawings which are:

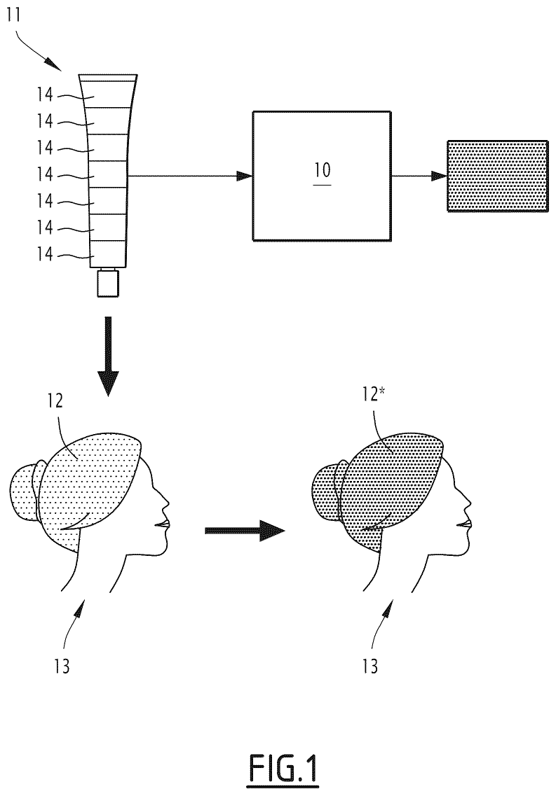

FIG. 1 is a schematic view of a concept of the first invention;

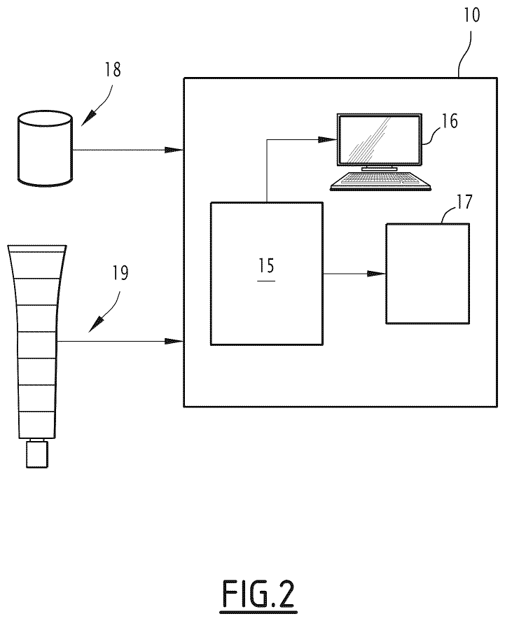

FIG. 2 is a schematic view of an electronic determination device according to the first invention;

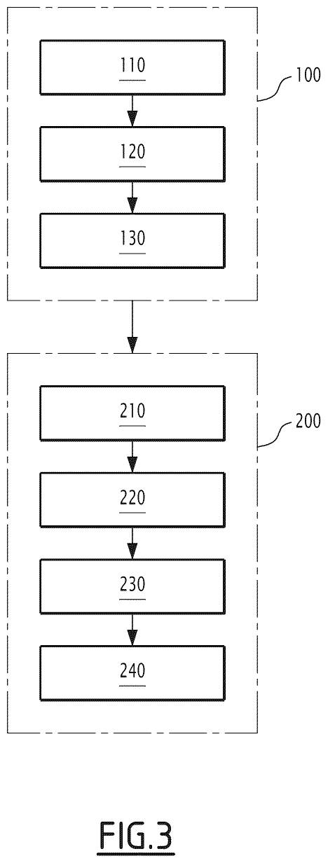

FIG. 3 is a flow chart of a determination method according to the first invention implemented by the electronic determination device of FIG. 2; and

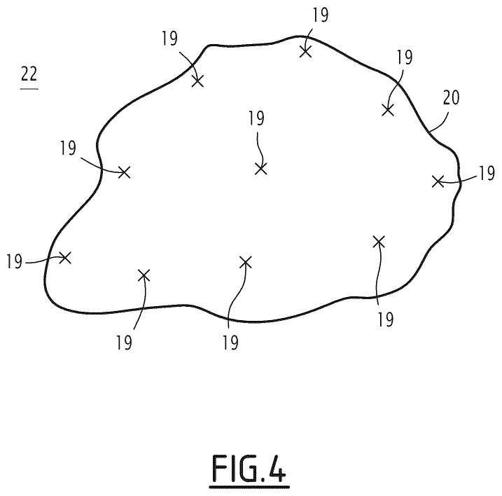

FIG. 4 is a two-dimensional schematic representation of a reduced viable cosmetic composition space.

In FIG. 1, an electronic device 10 for determining at least one colorimetric parameter associated with the cosmetic composition 11.

As seen in FIG. 1, the cosmetic composition 11 is a cosmetic composition for coloring hairs 12. Preferably, the cosmetic composition 11 is capable of being applied on the hair 12 of a user 13, to color said hair 12.

The cosmetic composition 11 comprises a plurality of ingredients 14 capable of interacting with each other, and with the hair 12 of the user 13, to color it.

Preferably, during this interaction, the ingredients 14 of the cosmetic composition 11 starts by decolorizing the hair 12. Then, the ingredients 14 of the cosmetic composition interact with the decolorized hair to fix pigments of the chosen color and thus form colored hair 12*.

The ingredients 14 are for example present in the cosmetic composition 11 in the form of powder, gel, emulsion or oil.

The electronic determination device 10 is configured to determine, using a set of magnitudes representing the quantity of each ingredient 14 in a cosmetic composition 11, a value of at least one colorimetric parameter characterizing the color of the hair 12 following the application of the cosmetic composition 11.

With reference to FIG. 2, the determination device 10 comprises a processing unit 15.

The determination device 10 optionally further comprises a display screen 16 and/or a unit 17 for producing cosmetic compositions 11.

The processing unit 15 comprises, for example, a calculator interacting with a computer program product. For example, the processing unit 15 is a computer.

The computer comprises, for example, a processor comprising a data processing unit, memories and an information medium reader, as well as optionally a human-machine interface.

The computer program product includes an information medium.

The information medium is a computer-readable medium, usually by the data processing unit. The readable data medium is a medium adapted to store electronic instructions and capable of being coupled with a computer system bus.

For example, the information medium is a USB flash disk, a floppy disk or flexible disk (“floppy disk”), an optical disk, a CD-ROM, a magnetic-optical disk, a ROM memory, a RAM memory, an EPROM memory, an EEPROM memory, a magnetic card or an optical card.

The computer program comprising program instructions is stored on the information medium.

The computer program can be loaded on the processing unit 15 and is adapted to implement a method for determining at least one colorimetric parameter, when the computer program is implemented on the processing unit of the computer. Such a determination method will be described hereinafter in the description.

The operation of the electronic device 10, implementing a method for determining at least one colorimetric parameter, will now be described with reference to the flow chart of FIG. 3, as well as the example of FIG. 4.

The determination method comprises a training phase 100.

The training phase 100 comprises a step 110 of receiving a set of viability constraint(s) of cosmetic composition(s) 11.

Preferably, each cosmetic composition(s) 11 comprises at least one ingredient 14 of a first type and at least one ingredient 14 of a second type.

Each ingredient 14 of the first type is for example a base, and each ingredient 14 of the second type is for example a coupler.

Preferably, each ingredient 14 is chosen from a predefined list of ingredients.

Preferably, the set of viability constraint(s) of cosmetic composition(s) 11 comprises one or more of the following constraints:
a ratio between a quantity of ingredient 14 of the first type and a quantity of ingredient 14 of the second type is between a first threshold and a second threshold,
a total quantity of the cosmetic composition 11 is less than a third threshold,
a quantity of each ingredient 14 in the cosmetic composition 11 is less than a fourth threshold,
a number of ingredients 14 in the cosmetic composition 11 is less than a fifth threshold.

Advantageously, the set of constraint(s) comprises each of the constraints cited above.

These constraints are optionally derived from chemical principles ensuring the viability of the cosmetic composition 11. The expression “viability of the cosmetic composition 11” is understood here to mean a composition complying with the set of constraint(s): is effective, and/or poses no risks for the user 13, and/or complies with production standards, and is preferably environmentally friendly.

The training phase 100 further comprises a step 120 of acquiring a set of training data 18.

Each training data item 18 of the set of training data 18 is specific to a cosmetic composition 11 complying with the set of viability constraint(s) received during the receiving step 110.

Each training data item 18 comprises a set of magnitudes representing the quantity of each ingredient 14 in the cosmetic composition 11, and a value of at least one colorimetric parameter associated with the cosmetic composition 11.

Each magnitude is for example a quantity of the corresponding ingredient 14 in the associated cosmetic composition 11.

Each quantity of ingredient 14 is optionally a quantity of substance indicated in mol, an ingredient mass, an ingredient volume, a mass percentage of the ingredient 14 in the cosmetic composition 11, or a volume percentage of the ingredient 14 in the cosmetic composition 11.

The or each colorimetric parameter characterizes the cosmetic composition 11 for coloring hair 12.

Preferably, each colorimetric parameter is representative of a visual effect of the hair 12 on which the cosmetic composition 11 is applied.

Advantageously, the at least one colorimetric parameter comprises:
a triplet of values characterizing a hair 12 color after applying the cosmetic composition 11, and/or
a color fade value of the cosmetic composition 11 after washing the hair 12, and/or
a selectivity value characterizing a difference in color between a root and a tip of the hair 12.

Preferably, each training data item 18 comprises each of the values cited above.

The triplet of values is for example is the CIE L*a*b* chromatic space value triplet.

The CIE L*a*b* color space, often abbreviated as CIELAB, is a color space for surface colors defined by the International Commission on Illumination (CIE) in 1976. It is based on evaluations of the CIE XYZ system, and abandons linearity to more accurately show up differences between colors perceived by the human eye. Three magnitudes characterize colors in this model, namely the lightness L* derived from the luminance (Y) of the XYZ evaluation, and two parameters a* and b* that express the color difference from the color of a gray surface with the same lightness, as the chrominance. The definition of a gray, uncolored, achromatic surface implies that the composition of the light that illuminates the colored surface is explicitly indicated. This illuminant is often daylight corresponding to the D65 normalized standard.

Also, in this case, the triplet of values comprises a lightness value L*, and two color difference values a*, b* from the color of a gray surface with the same lightness.

Any other color space could be envisaged.

Alternatively, the triplet of values is an RGB triplet. The triplet then comprises a value R for the red color, a value G for green and a value B for blue.

More generally, any representations making it possible to associate spectra could also be used in this context.

The color of a hair is not identical at all points of the hair. In particular, the tips of hair 12 are generally lighter than the roots. Thus, the selectivity value is parameter of interest.

Preferably, during the acquisition step 120 of the training phase 100, the set of training data 18 is acquired, at least in part, via a sensor capable of measuring the at least one colorimetric parameter.

The sensor is for example a spectrocolorimeter.

According to an embodiment, the sensor is capable of capturing images of zones of hairs of the individual and extracting the colorimetric measurements from the images captured.

Preferably, each training data item 18 is derived from a viable cosmetic composition 11, i.e., complying with the set of viability constraint(s).

Preferably, each training data item corresponds to a marketed cosmetic composition. Thus, before the marketing of said cosmetic compositions, their colorimetric parameters have been measured during testing on hair 12.

Furthermore and optionally, the cosmetic compositions associated with the training data are preselected by expert chemists from all the marketed cosmetic compositions.

The training phase 100 further comprises a step 130 of training an artificial intelligence model using the set of training data 18, to obtain a trained model.

Preferably, the artificial intelligence model comprises at least one of the following models: a support vector machine, a random forest, a gradient boosting mechanism, or kriging, also known as Gaussian process regressor.

In each of the above cases, the artificial intelligence model comprises adjustable parameters. During the training step 130, each adjustable parameter is adjusted such that, when a respective set of magnitudes of a training data item 18 is given as an input, said trained model supplies as an output, one or more colorimetric parameters substantially identical to the colorimetric parameter(s) of said training data item 18.

The determination method further comprises a processing phase 200 during which the trained model is used to determine colorimetric parameters associated with one or more colorimetric compositions under test.

The processing phase 200 comprises a step 210 of obtaining test data item(s) 19 relating to a cosmetic composition 11 under test.

The test data item 19, for each cosmetic composition 11 under test, a set of magnitudes representing the quantity of each ingredient 14 in the cosmetic composition under test.

Preferably, the ingredients 14 of each cosmetic composition 11 under test are chosen from the predefined list of ingredients 14 described above.

Each cosmetic composition 11 under test complies with the set of viability constraint(s) received during the training phase 100.

The obtaining step 210 advantageously comprises receiving a number N corresponding to a number of cosmetic compositions 11 under test. Optionally, the number N is greater than or equal to two. Preferably, the number N is greater than one hundred, for example equal to one hundred and fifty.

The obtaining step 210 furthermore advantageously comprises generating N test data items 19. Each test data item 19 relates to a cosmetic composition 11 of which the ingredients 14 are chosen from the predefined list of ingredients 14 and such that the cosmetic composition 11 associated with each test data item 19 is within a reduced viable cosmetic composition space 20. The reduced viable cosmetic composition space 20 only comprises cosmetic compositions 11 complying with the set of viability constraint(s). The test data items 19 are representative of the reduced viable cosmetic composition space 20.

The expression “test data items 19 representative of the reduced space 20” is understood here to mean that the test data items 19 are aptly chosen in the reduced space 20 to substantially cover the entire reduced space 20.

FIG. 4 illustrates, via a two-dimensional block diagram, a set 22 representing all the possible cosmetic compositions 11 and the reduced space 20. It is clear that this set 22 and this space 20 cannot, in fact, be represented two-dimensionally but rather in a number of dimensions equal to the number of ingredients 14 in the predefined list of ingredients.

In FIG. 4, the set 22 of all the possible cosmetic compositions 11 corresponds to the map in FIG. 4. As seen in FIG. 4, the reduced space 20 is included in said set 22 because the reduced space 20 only comprises the cosmetic compositions 11 of the set 20 complying with the viability constraint(s).

Again in FIG. 4, the test data items 19 in the reduced space 20 can be seen. For example, ten test data items 19 are represented in FIG. 4.

Preferably, generating N test data items 19 comprises computing magnitudes of the N test data items 19 such that, for each test data item 19, the distance between said test data item 19 and the other test data items 19 is maximum.

Each magnitude of each test data item 19 represents the quantity of an ingredient 14 in the corresponding cosmetic composition 11 under test.

Generating N test data items 19 comprising computing magnitudes of N sets of magnitudes for which a distance between each pair of sets of magnitudes is maximum. Each magnitude of a respective set of magnitudes represents the quantity of an ingredient 14 in a cosmetic composition 11 associated with said set of magnitudes, said cosmetic composition 11 complying with the set of constraint(s). For example, a test data item 19 comprises a first magnitude equal to 4 mol of the first ingredient 14A and 6 mol of a second ingredient 14B.

Advantageously, each test data item 19 is a vector comprising a coefficient for each ingredient 14 of the predefined list of ingredients.

Each coefficient represents the quantity of said ingredient 14 in the respective cosmetic composition 11 of the associated vector.

A distance between two test data items 19, i.e., between two vectors, is for example defined by the algebraic norm according to the following equation:

$$distance ( X , Y ) = ‖ X - Y ‖ = \sqrt{{\sum }_{i = 1}^{P} {( {x}_{i} - {y}_{i} )}^{2}} \qquad [ MATH 1 ]$$

where X and Y are two sets of P magnitudes,
x_{i,i=1, . . . ,P} and y_{i,i=1, . . . ,P} are the respective magnitudes of said sets X and Y,
√ is the square root function, and
Σ Is the sum operator.

The N vectors are computed for example by applying a design of experimentation technique, also known as DoE technique. Advantageously, the DoE technique implements an SFD (space filling design) algorithm. This algorithm makes it possible, even with a reduced number of vectors, to ensure that the vectors chosen are representative of the reduced space 20. Indeed, when the number of vectors is low, the hypotheses of the law of large numbers are not sufficiently fulfilled for a random distribution of vectors to be able to be representative of the reduced space 20.

In FIG. 4, it can be seen that the test data items 19 are spaced apart from one another so as to maximize the distances between the test data items 19.

Alternatively, the obtaining step 210 only comprises acquiring one or more test data items 19 chosen by an operator of the determination device 10. In this case, the operator chooses one or more cosmetic compositions by selecting, for each cosmetic composition, ingredients 14 from the predefined list of ingredients, and the quantity of each of said ingredients 14. When choosing, the operator ensures that each cosmetic composition 11 chosen complies with the set of constraint(s).

The processing phase 200 further comprises a step 220 of applying, to the test data items obtained 19, the trained model to determine the at least one colorimetric parameter of the or each associated cosmetic composition 11.

Preferably, during the application step 220, each test data item 19 is supplied successively to the trained model. The trained model then determines, for each test data item 19, the associated colorimetric parameter(s).

Advantageously, during the application step 220, the cosmetic composition 11 and the colorimetric parameter(s) associated with each test data item are stored in the memory or memories of the processing unit 15.

Furthermore and optionally, the processing phase 200 further comprises a step 230 of sending the or each colorimetric parameter and the associated test data item, to the display screen 16. The display screen 16 then displays for each test data item, a rendering representative of the application, to hair 12, of the cosmetic composition.

Preferably, during the sending step 230, the display screen displays an image of a sample of hair 12 to which the cosmetic composition associated with each test data item is applied. Each image is computed using the colorimetric parameter(s).

If the colorimetric parameters comprise the triplet of values characterizing a color of the hair 12 after applying the cosmetic composition 11, the display screen displays a first image of a sample of hair 12 to which the cosmetic composition associated with each test data item is applied.

If the colorimetric parameters further comprise a color face value of the cosmetic composition 11 after washing the hair 12, the display screen 16 furthermore displays for example a second image of the same sample of hair 12 after washing the hair 12, computed using the fade value.

If the colorimetric parameters additionally, or instead, comprise the selectivity value, the display screen displays for example a third image of a zoom of the sample of hair 12 illustrating the color difference between the root and the tip of the hair 12.

According to another optional addition, the processing phase 200 further comprises a step 240 of producing at least one sample of cosmetic composition 11 under test. The at least one sample is preferably produced by the unit 17 for producing cosmetic compositions 11.

For example, during the production step 240, a sample of the cosmetic composition 11 associated with each test data item is produced.

Alternatively, the production step 240 comprises receiving, from the operator, a selection of one or more cosmetic compositions 11 to be produced, from the cosmetic compositions 11 under test. The production unit 17 then produces only the sample(s) of the cosmetic compositions 11 selected by the operator.

The sample(s) of cosmetic compositions 11 produced are intended to be applied on hair 12, preferably a lock of hair 12, to verify the colorimetric parameter(s).

Thus, following the application of each sample on hair 12, the colorimetric parameters are measured using the same sensors used to form the training data 18.

Alternatively, the processing phase 200 does not comprise the sending step 230 and/or the production step 240.

Also alternatively, each cosmetic composition 11 is capable of being applied to any type of hairs 12, and not only hair, for example to eyelashes, eyebrows or a beard of the user 13.

With the determination method described above, the development of novel cosmetic compositions 11 is accelerated since it is possible to determine digitally the colorimetric parameter(s) associated with a cosmetic composition 11 without needing to test it physically.

Furthermore, the method makes it possible to obtain color more adapted to the user's wishes, since a large number of cosmetic compositions can be tested rapidly.

Furthermore, the fact that the test data items 19 are representative of the reduced space 20 makes it possible to discover novel cosmetic compositions 11 which have not been hitherto envisaged. Furthermore, this makes it possible to obtain a substantially general idea of the different possibilities of cosmetic compositions 11 complying with the set of constraint(s).

All the variants and optional additions listed above can be combined with each other.

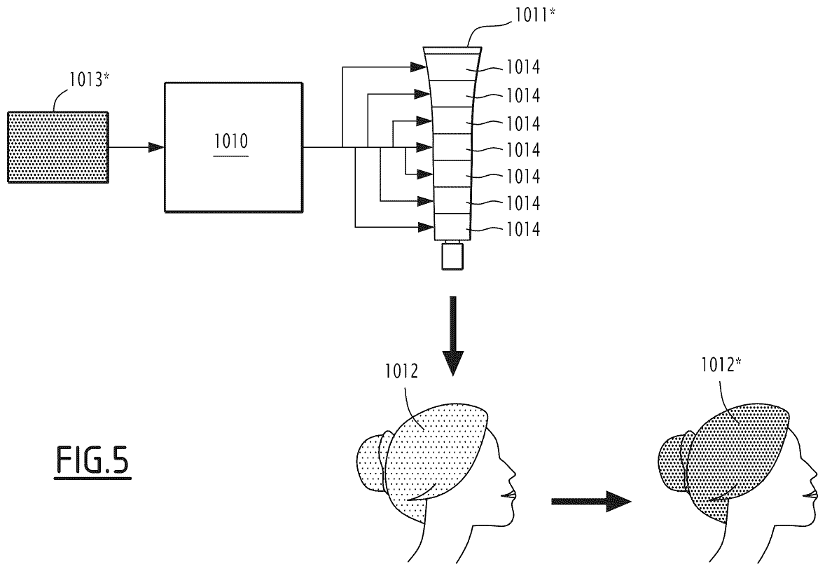

A second invention will now be described with reference to FIGS. 5 to 9.

The second invention relates to a method for determining a target cosmetic composition for coloring hairs, particularly hair, according to at least one target colorimetric parameter. The present second invention also relates to an associated computer program product and electronic determination device.

The present second invention relates to the field of cosmetic products, preferably cosmetic compositions for coloring hairs, in particular hair.

More generally, a “cosmetic product” is a product as defined in Regulation EC No. 1223/2009 of the European Parliament and of the Council of Nov. 30, 2009, relating to cosmetic products.

An objective of the cosmetic industry is to improve the experience of its consumers.

There is in particular a strong trend to propose products that are increasingly adapted to the needs and characteristics specific to the user. This trend is generally called “customization”.

Customizing cosmetic products and services can concern any part of the human body but is of particular interest for exposed body parts, such as the face (makeup or care products), and the hair or beard where applicable (coloring products for example).

In the field of cosmetic compositions for coloring hair(s), in particular hair, novel cosmetic compositions are regularly developed to adapt to user demand.

These novel cosmetic compositions are aimed, in a first case, at achieving a novel color or a novel visual effect of the hairs on which they are applied. In a second case, these novel cosmetic compositions are aimed at achieving a previously known color but comprise ingredients not previously used for this purpose. This second case is of particular interest when the procurement of certain ingredients becomes difficult, is accompanied by high costs, or poses a risk for the environment.

However, obtaining novel cosmetic compositions results from a lengthy and complex process.

Firstly, expert chemists develop cosmetic composition prototypes. These prototypes result from the experience of the expert chemists and are governed by certain chemical principles.

Then, these prototypes are tested individually in a laboratory to check that the visual effect obtained meets the expert chemists' expectations.

In some cases and unpredictably, the visual effect associated with a newly developed prototype does not meet expectations. It is therefore not possible to capitalize on the work of the expert chemists who developed these prototypes.

Furthermore, it is sometimes sought to obtain several cosmetic compositions resulting in the same color or the same visual effect. This makes it possible to pre-empt future issues due to the unavailability of certain ingredients, while ensuring to continue being able to meet demand from users seeking to obtain said color or said visual effect.

Therefore, there is a need for tools making it possible to simplify the development of a novel cosmetic composition.

To this end, the present second invention relates to a method for determining a target cosmetic composition for coloring hairs, in particular hair, according to at least one target colorimetric parameter, the method being implemented by an electronic determination device and comprising a processing phase comprising the following steps:
acquiring the at least one target colorimetric parameter,
obtaining a set of cosmetic compositions under test, each cosmetic composition under test comprising ingredients,
obtaining at least one colorimetric parameter associated with each cosmetic composition under test,
filtering the cosmetic compositions under test according to the at least one colorimetric parameter associated with them, to obtain filtered cosmetic compositions, the at least one colorimetric parameter associated with each filtered cosmetic composition meeting a criterion relating to the at least one target colorimetric parameter,
forming several clusters of filtered cosmetic compositions such that the filtered cosmetic compositions from the same cluster comprise the same ingredients,
selecting a cluster of filtered cosmetic compositions, referred to as selected cluster, and
determining the target cosmetic composition according to the filtered cosmetic compositions of the selected cluster.

Thanks to the steps of filtering and of forming clusters of cosmetic compositions, it is possible to cluster together the cosmetic compositions of which at least one colorimetric parameter is similar to the at least one target colorimetric parameter and of which the ingredients are identical. Thus, it is possible to rapidly determine one or more cosmetic compositions of which the at least one colorimetric parameter corresponds to the target colorimetric parameter(s).

According to particular embodiments, the determination method according to the second invention comprises one or several of the following characteristics, taken in isolation or in any technically possible combination:
each filtered cosmetic composition comprises, for each of its ingredients, a quantity of said ingredient in the filtered cosmetic composition, the determination step of the processing phase comprising:
   for each ingredient of the filtered cosmetic compositions of the selected cluster, computing an average value of the quantities of said ingredient from said filtered cosmetic compositions,
   determining an average cosmetic composition comprising, for each of said ingredients, a quantity equal to the respective computed average value of the ingredient, and
   forming the target cosmetic composition according to the average cosmetic composition;
during the determination step of the processing phase, forming the target cosmetic composition comprises computing at least one colorimetric parameter associated with the average cosmetic composition and optimizing the quantity of each ingredient of the average cosmetic composition such that the at least one colorimetric parameter associated with the average cosmetic composition approaches the at least one target colorimetric parameter, the target cosmetic composition being the average cosmetic composition resulting from optimizing its quantities of ingredients;
during the selection step of the processing phase, M clusters of filtered cosmetic compositions are selected, M being greater than or equal to two, during the determination step of the processing phase, for each selected cluster, a corresponding target cosmetic composition being determined;
during the selection step of the processing phase, the M clusters are selected such that, for each selected cluster, the ingredients comprised in the filtered cosmetic compositions of said selected cluster are only comprised in said selected cluster;
the method comprises, prior to the processing phase, a training phase including the following steps:
   receiving a set of viability constraint(s) of cosmetic composition(s),
   acquiring a set of training data, each training data item being specific for a cosmetic composition complying with the set of viability constraint(s) received,
each training data item comprising:
   a set of magnitudes representing the quantity of each ingredient of the cosmetic composition, and
   a value of the at least one colorimetric parameter associated with the cosmetic composition, and
   training an artificial intelligence model based on the set of training data, to obtain a trained model,
during the step of obtaining at least one colorimetric parameter associated with each cosmetic composition under test, of the processing phase, each colorimetric parameter being obtained by applying the trained model to a corresponding cosmetic composition under test;
the step of obtaining a set of cosmetic compositions under test, of the processing phase comprises:
   receiving a number N corresponding to the number of cosmetic compositions under test, the number N being greater than or equal to two,
   generating N test data items,
each test data item relating to a cosmetic composition of which the ingredients are chosen from a predefined list of ingredients and such that the cosmetic composition associated with each test data item is within a reduced viable cosmetic composition space, the reduced space only comprising the cosmetic compositions complying with a set of viability constraint(s), and the N test data items being representative of said reduced viable cosmetic composition space,
the set of cosmetic compositions under test being formed from the N test data items generated;
during the processing phase, generating N test data items of the obtaining step comprises computing magnitudes of the N test data items such that, for each test data item, the distance between said test data item and the other test data items is maximum,
each magnitude of each test data item representing the quantity of an ingredient in the corresponding cosmetic composition under test
the at least one colorimetric parameter comprises:
   a triplet of values characterizing a hair color after applying the cosmetic composition, or
   a color fade value of the cosmetic composition after washing the hairs, or
   a selectivity value characterizing a difference in color between a root and a tip of the hairs; and
the processing phase further comprises a step of producing at least one sample of the target cosmetic composition, with a view to applying it on hairs to verify the value of the at least one target colorimetric parameter.

The present second invention also relates to a computer program product on which a computer program comprising program instructions is stored, the computer program being loaded onto a data processing unit and implementing such a method when the computer program is implemented on the data processing unit.

The present second invention also relates to an electronic device for determining a target cosmetic composition for coloring hairs, in particular hair, according to at least one target colorimetric parameter, the electronic determination device comprising a processing unit capable of implementing such a method.

The present second invention also relates to a readable information medium on which a computer program product comprising program instructions is stored, the computer program being loaded onto a data processing unit and implementing such a determination method when the computer program is implemented on the data processing unit.

Other features and advantages of the second invention will appear when reading the following description of embodiments of the second invention, provided solely as an example and with reference to the drawings which are:

FIG. 5 is a schematic view of a concept of the second invention;

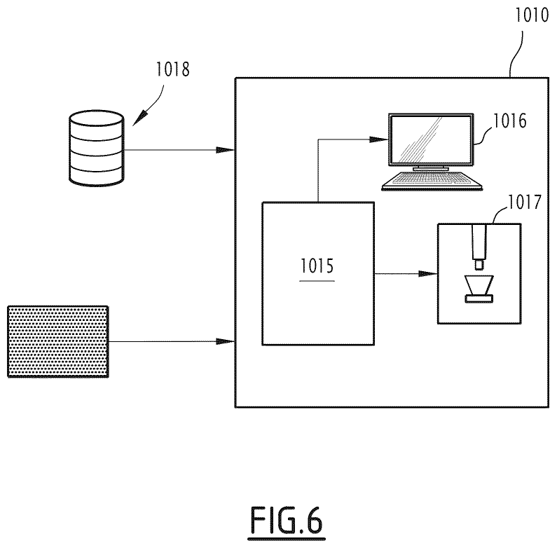

FIG. 6 is a schematic view of an electronic determination device according to the second invention;

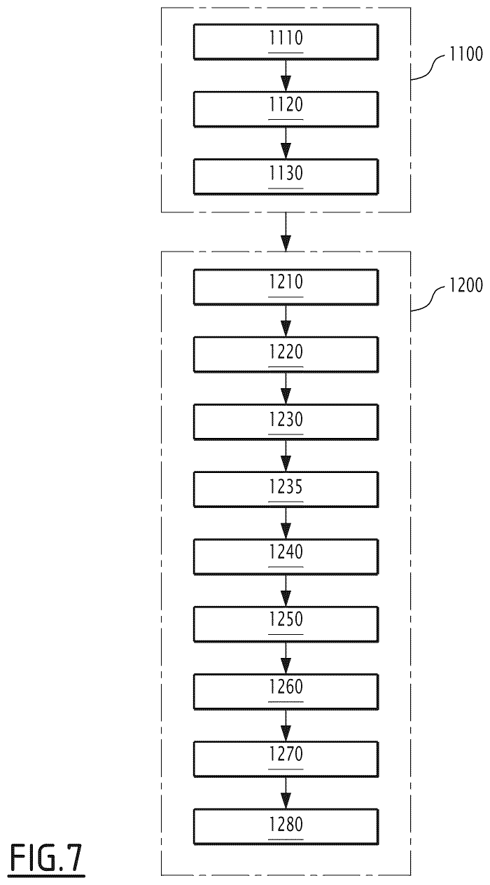

FIG. 7 is a flow chart of a determination method according to the second invention implemented by the electronic determination device of FIG. 6;

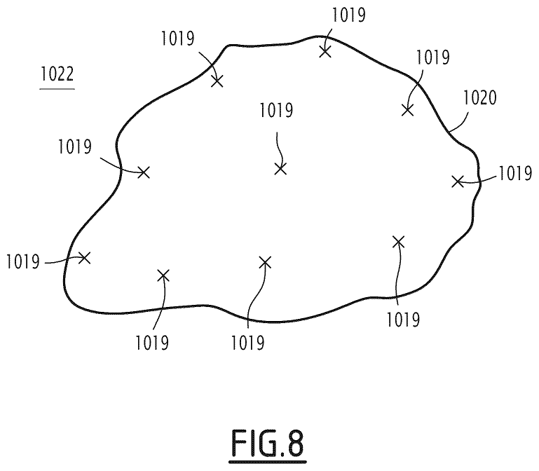

FIG. 8 is a two-dimensional schematic representation of a reduced viable cosmetic composition space; and

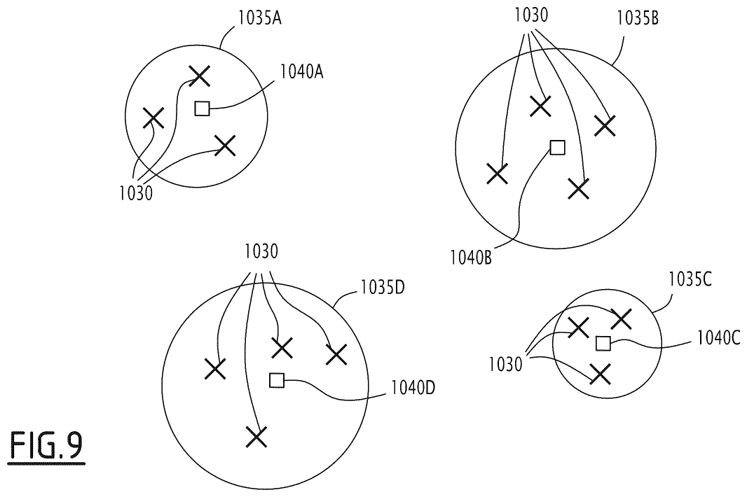

FIG. 9 is a two-dimensional schematic representation of the determination method illustrated in FIG. 3.

In FIG. 9, an electronic device 1010 for determining a target cosmetic composition 1011* for coloring hairs 1012, in particular hair, according to at least one target colorimetric parameter 1013*, is represented.

As seen in FIG. 9, the target cosmetic composition 11* is a cosmetic composition for coloring hairs 1012. Preferably, the target cosmetic composition 11* is capable of being applied on the hair 1012 of a user, to color said hair 1012.

The target cosmetic composition 1011* comprises a plurality of ingredients 1014 capable of interacting with each other, and with the hair 1012 of the user, to color it.

Preferably, during this interaction, the ingredients 1014 of the target cosmetic composition 1011* start by decolorizing the hair 1012. Then, the ingredients 1014 of the cosmetic composition 1011 interact with the decolorized hair to fix pigments of the chosen color and thus form colored hair 1012*.

The colored hair 1012* has a color corresponding to the at least one target colorimetric parameter 1013*.

The ingredients 1014 are for example present in the target cosmetic composition 1011* in the form of powder, gel, emulsion or oil.

The electronic determination device 1010 is configured to determine, using the at least one target colorimetric parameter 1013*, a set of magnitudes representing the quantity of each ingredient 1014 forming a target cosmetic composition 1011*. The target cosmetic composition 1011* is intended to produce a hair coloring corresponding to the at least target colorimetric parameter 1013*, when it is applied on the user's hair 1012.

With reference to FIG. 6, the determination device 1010 comprises a processing unit 1015.

The determination device 1010 optionally further comprises a display screen 1016 and/or a unit 1017 for producing cosmetic compositions 1011.

The processing unit 1015 comprises, for example, a calculator interacting with a computer program product. For example, the processing unit 1015 is a computer.

The computer comprises, for example, a processor comprising a data processing unit, memories and an information medium reader, as well as optionally a human-machine interface.

The computer program product includes an information medium.

The information medium is a computer-readable medium, usually by the data processing unit. The readable data medium is a medium adapted to store electronic instructions and capable of being coupled with a computer system bus.

For example, the information medium is a USB flash disk, a floppy disk or flexible disk (“floppy disk”), an optical disk, a CD-ROM, a magnetic-optical disk, a ROM memory, a RAM memory, an EPROM memory, an EEPROM memory, a magnetic card or an optical card.

The computer program comprising program instructions is stored on the information medium.

The computer program can be loaded on the processing unit 1015 and is adapted to implement a method for determining at least one target cosmetic composition 1011*, when the computer program is implemented on the processing unit of the computer. Such a determination method will be described hereinafter in the description.

The operation of the electronic device 1010, implementing a method for determining a target cosmetic composition 1011*, will now be described with reference to the flow chart of FIG. 7, as well as the examples of FIGS. 8 and 9.

The determination method optionally comprises a training phase 1100.

The training phase 1100 comprises a step 1110 of receiving a set of viability constraint(s) of cosmetic composition(s) 1011.

Preferably, each cosmetic composition(s) 1011 comprises at least one ingredient 1014 of a first type and at least one ingredient 1014 of a second type.

Each ingredient 1014 of the first type is for example a base, and each ingredient 1014 of the second type is for example a coupler.

Preferably, each ingredient 1014 is chosen from a predefined list of ingredients. Preferably, the set of viability constraint(s) of cosmetic composition(s) 1011 comprises one or more of the following constraints:
a ratio between a quantity of ingredient 1014 of the first type and a quantity of ingredient 1014 of the second type is between a first threshold and a second threshold,
a total quantity of the cosmetic composition 1011 is less than a third threshold,
a quantity of each ingredient 1014 in the cosmetic composition 1011 is less than a fourth threshold,
a number of ingredients 1014 in the cosmetic composition 1011 is less than a Advantageously, the set of constraint(s) comprises each of the constraints cited above.

These constraints are optionally derived from chemical principles ensuring the viability of the cosmetic composition 1011. The expression “viability of the cosmetic composition 1011” is understood here to mean a composition complying with the set of constraint(s): is effective, and/or poses no risks for the user 1013, and/or complies with production standards, and is preferably environmentally friendly.

The optional training phase 1100 further comprises a step 1120 of acquiring a set of training data 1018.

Each training data item 1018 of the set of training data 1018 is specific to a cosmetic composition 1011 complying with the set of viability constraint(s) received during the receiving step 1110.

Each training data item 1018 comprises a set of magnitudes representing the quantity of each ingredient 1014 in the cosmetic composition 1011, and a value of at least one colorimetric parameter 1013 associated with the cosmetic composition 1011.

Each magnitude is for example a quantity of the corresponding ingredient 1014 in the associated cosmetic composition 1011.

Each quantity of ingredient 1014 is optionally an ingredient mass, an ingredient volume, a mass percentage of the ingredient 1014 in the cosmetic composition 1011, or a volume percentage of the ingredient 1014 in the cosmetic composition 1011.

The or each colorimetric parameter 1013 characterizes the cosmetic composition 1011 for coloring hair 1012.

Preferably, each colorimetric parameter 1013 is representative of a visual effect of the hair 1012 on which the cosmetic composition 1011 is applied.

Advantageously, the at least one colorimetric parameter 1013 comprises:
a triplet of values characterizing a hair 1012 color after applying the cosmetic composition 1011, and/or
a color fade value of the cosmetic composition 1011 after washing the hair 1012, and/or
a selectivity value characterizing a difference in color between a root and a tip of the hair 1012.

Preferably, each training data item 1018 comprises each of the values cited above.

The triplet of values is for example is the CIE L*a*b* chromatic space value triplet.

The CIE L*a*b* color space, often abbreviated as CIELAB, is a color space for surface colors defined by the International Commission on Illumination (CIE) in 1976. It is based on evaluations of the CIE XYZ system, and abandons linearity to more accurately show up differences between colors perceived by the human eye. Three magnitudes characterize colors in this model, namely the lightness L* derived from the luminance (Y) of the XYZ evaluation, and two parameters a* and b* that express the color difference from the color of a gray surface with the same lightness, as the chrominance. The definition of a gray, uncolored, achromatic surface implies that the composition of the light that illuminates the colored surface is explicitly indicated. This illuminant is often daylight corresponding to the D65 normalized standard.

Also, in this case, the triplet of values comprises a lightness value L*, and two color difference values a*, b* from the color of a gray surface with the same lightness. Any other color space could be envisaged.

Alternatively, the triplet of values is an RGB triplet. The triplet then comprises a value R for the red color, a value G for green and a value B for blue.

More generally, any representations making it possible to associate spectra could also be used in this context.

The color of a hair is not identical at all points of the hair. In particular, the tips of hair 1012 are generally lighter than the roots. Thus, the selectivity value is parameter of interest.

Preferably, during the acquisition step 1120 of the training phase 1100, the set of training data 1018 is acquired, at least in part, via a sensor capable of measuring the at least one colorimetric parameter 1013.

The sensor is for example a spectrocolorimeter.

According to an embodiment, the sensor is capable of capturing images of zones of hairs of the individual and extracting the colorimetric measurements from the images captured.

Preferably, each training data item 1018 is derived from a viable cosmetic composition 1011, i.e., complying with the set of viability constraint(s).

Preferably, each training data item corresponds to a marketed cosmetic composition. Thus, before the marketing of said cosmetic compositions, their colorimetric parameters 1013 have been measured during testing on hair 1012.

Furthermore and optionally, the cosmetic compositions associated with the training data are preselected by expert chemists from all the marketed cosmetic compositions.

The optional training phase 1100 further comprises a step 1130 of training an artificial intelligence model using the set of training data 1018, to obtain a trained model.

Preferably, the artificial intelligence model comprises at least one of the following models: a support vector machine, a random forest, a gradient boosting mechanism, or kriging, also known as Gaussian process regressor.

In each of the above cases, the artificial intelligence model comprises adjustable parameters. During the training step 1130, each adjustable parameter is adjusted such that, when a respective set of magnitudes of a training data item 1018 is given as an input, said trained model supplies as an output, one or more colorimetric parameters 1013 substantially identical to the colorimetric parameter(s) of said training data item 1018.

It is clear that the aim of the training phase 1100 described above is to obtain the trained model.

According to a variant not shown, the determination method does not comprise the training phase 1100.

For example, the method comprises, instead, a step of receiving the trained model. According to another example, the method comprises nothing instead.

The method further comprises a processing phase 1200.

The processing phase 1200 comprises a step 1210 of acquiring the at least one target colorimetric parameter 1013*. The at least one target colorimetric parameter 1013* is for example supplied by an operator of the determination device 10.

The processing phase 1200 further comprises a step 1220 of obtaining a set of cosmetic compositions 1011 under test.

The set of cosmetic compositions 1011 under test is preferably formed from a plurality of test data items 1019.

The test data item 1019, for each cosmetic composition 1011 under test, a set of magnitudes representing the quantity of each ingredient 1014 in the cosmetic composition 1011 under test.

Preferably, the ingredients 1014 of each cosmetic composition 1011 under test are chosen from the predefined list of ingredients 1014 described above.

Each cosmetic composition 1011 under test complies with the set of viability constraint(s) received during the training phase 1100.

If the method does not comprise the training phase 1100, then the step 1220 of obtaining the set of cosmetic compositions 1011 under test further comprises receiving said set of viability constraint(s).

The step 1220 of obtaining the set of cosmetic compositions 1011 under test advantageously comprises receiving a number N corresponding to a number of cosmetic compositions 1011 under test. Optionally, the number N is greater than or equal to two. Preferably, the number N is greater than one hundred, for example equal to one hundred and fifty.

The obtaining step 1220 furthermore advantageously comprises generating N test data items 1019. Each test data item 1019 relates to a cosmetic composition 1011 of which the ingredients 1014 are chosen from the predefined list of ingredients 1014 and such that the cosmetic composition 1011 associated with each test data item 1019 is within a reduced viable cosmetic composition space 1020. The reduced viable cosmetic composition space 1020 only comprises cosmetic compositions 1011 complying with the set of viability constraint(s). The test data items 1019 are representative of the reduced viable cosmetic composition space 1020.

The expression “test data items 1019 representative of the reduced space 1020” is understood here to mean that the test data items 1019 are aptly chosen in the reduced space 1020 to substantially cover the entire reduced space 1020.

FIG. 8 illustrates, via a two-dimensional block diagram, a set 1022 representing all the possible cosmetic compositions 1011 and the reduced space 1020. It is clear that this set 1022 and this space 1020 cannot, in fact, be represented two-dimensionally but rather in a number of dimensions equal to the number of ingredients 1014 in the predefined list of ingredients.

In FIG. 8, the set 1022 of all the possible cosmetic compositions 1011 corresponds to the map in FIG. 8. As seen in FIG. 8, the reduced space 1020 is included in said set 1022 because the reduced space 1020 only comprises the cosmetic compositions 1011 of the set 1020 complying with the viability constraint(s).

Again in FIG. 8, the test data items 1019 in the reduced space 1020 can be seen. For example, ten test data items 1019 are represented in FIG. 8.

Preferably, generating N test data items 1019 comprises computing magnitudes of the N test data items 1019 such that, for each test data item 1019, the distance between said test data item 1019 and the other test data items 1019 is maximum.

Each magnitude of each test data item 1019 represents the quantity of an ingredient 1014 in the corresponding cosmetic composition 1011 under test.

Generating N test data items 1019 comprising computing magnitudes of N sets of magnitudes for which a distance between each pair of sets of magnitudes is maximum. Each magnitude of a respective set of magnitudes represents the quantity of an ingredient 1014 in a cosmetic composition 1011 associated with said set of magnitudes, said cosmetic composition 1011 complying with the set of constraint(s). For example, a test data item 1019 comprises a first magnitude equal to 4 mol of the first ingredient 1014A and 6 mol of a second ingredient 1014B.

Advantageously, each test data item 1019 is a vector comprising a coefficient for each ingredient 1014 of the predefined list of ingredients.

Each coefficient represents the quantity of said ingredient 1014 in the respective cosmetic composition 1011 of the associated vector.

A distance between two test data items 1019, i.e., between two vectors, is for example defined by the algebraic norm according to the following equation:

$$distance ( X , Y ) = ‖ X - Y ‖ = \sqrt{{\sum }_{i = 1}^{P} {( {x}_{i} - {y}_{i} )}^{2}} \qquad [ MATH 1 ]$$

where X and Y are two sets of P magnitudes,
x_{i,i=1, . . . ,P} and y_{i,i=1, . . . ,P} are the respective magnitudes of said sets X and Y,
√ is the square root function, and
Σ Is the sum operator.

The N vectors are computed for example by applying a design of experimentation technique, also known as DoE technique. Advantageously, the DoE technique implements an SFD (space filling design) algorithm. This algorithm makes it possible, even with a reduced number of vectors, to ensure that the vectors chosen are representative of the reduced space 1020. Indeed, when the number of vectors is low, the hypotheses of the law of large numbers are not sufficiently fulfilled for a random distribution of vectors to be able to be representative of the reduced space 1020.

In FIG. 8, it can be seen that the test data items 1019 are spaced apart from one another so as to maximize the distances between the test data items 1019.

Alternatively, the obtaining step 1220 only comprises acquiring one or more test data items 1019 chosen by an operator of the determination device 1010. In this case, the operator chooses one or more cosmetic compositions by selecting, for each cosmetic composition 1011, ingredients 1014 from the predefined list of ingredients, and the quantity of each of said ingredients 1014. When choosing, the operator ensures that each cosmetic composition 1011 chosen complies with the set of constraint(s).

The set of cosmetic compositions under test is formed by the N test data items 1019.

The processing phase 1200 further comprises a step 1230 of obtaining at least one colorimetric parameter 1013 associated with each cosmetic composition 1011 under test.

Preferably, the obtaining step 1230 comprises applying, to the test data items 1019, forming the set of cosmetic compositions 1011 obtained, the trained model to determine the at least one colorimetric parameter 13 of the or each cosmetic composition 1011 under test.

Preferably, during the obtaining step 1230, each test data item 1019 is supplied successively to the trained model. The trained model then determines, for each test data item 1019, the associated colorimetric parameter(s) 1013.

Advantageously, during the obtaining step 1230, the cosmetic composition 1011 and the colorimetric parameter(s) 1013 associated with each test data item are stored in the memory or memories of the processing unit 1015.

Alternatively, the obtaining step 1230 comprises receiving, from the operator of said device 1010, at least one colorimetric parameter 1013 associated with each cosmetic composition 1011 of the set of cosmetic compositions 1011 under test.

In this case, said at least one colorimetric parameter 1013 is for example measured by the operator of the determination device 1010 for example by means of a sensor, as explained above for the test data items 1019.

This is particularly advantageous if the method does not comprise the training phase 1100, or reception of the trained model.

This variant is combined particularly advantageously with the variant of the step 1220 of obtaining the set of cosmetic compositions 1011 under test, whereby the test data items 1019 associated with the cosmetic compositions 1011 under test are received from the operator.

The processing phase 1200 further comprises a filtering step 1235, during which the cosmetic compositions 1011 under test are filtered according to the at least one colorimetric parameter 1013 associated with them. The filtering step 1235 makes it possible to obtain filtered cosmetic compositions 1030, for which the at least one colorimetric parameter 1013 meets a criterion relating to the at least one target colorimetric parameter 1013*.

Preferably, the criterion is that a relative difference between the at least one colorimetric parameter 1013 associated with each cosmetic composition 1011 under test and the at least one target colorimetric parameter 1013* is less than a sixth threshold. The sixth threshold is for example equal to 10%. The filtered cosmetic compositions 1030 are only the cosmetic compositions 1011 under test for which said relative difference is less than the sixth threshold. In other words, the filtered cosmetic compositions 1030 are the cosmetic compositions 1011 under test for which each colorimetric parameter 1013 deviates, from the at least one target colorimetric parameter 1013*, by not more than the sixth threshold.

The processing phase 1200 further comprises a step 1240 of forming several clusters 1035 of filtered cosmetic compositions 1030. For each cluster 1035, the filtered cosmetic compositions 1030 comprise the same ingredients 1014. The formation step 1240 comprises for example the application of an unsupervised learning algorithm, such as a k-means algorithm.

The filtered cosmetic compositions 1030 of each cluster 1035 are substantially similar. In other words, two filtered cosmetic compositions 1030 comprising the same ingredients 1014 but in substantially different proportions are not comprised in the same cluster 1035. For example, a filtered cosmetic composition 1030 comprising 10% of an ingredient 1014A and 90% of an ingredient 1014B is not comprised in the same cluster 1035 as a filtered cosmetic composition 1030 comprises 80% of the ingredient 1014A and 20% of the ingredient 1014B.

In FIG. 9, several clusters 1035A, 1035B, 1035C, 1035D are represented by ellipses. The filtered cosmetic compositions 1030 of each cluster 1035A, 1035B, 1035C, 1035D are represented by crosses.

As seen in FIG. 9, a first cluster 1035A comprises the filtered cosmetic compositions 1030 formed from the ingredients 1014A and 1014B. A second cluster 1035B comprises the filtered cosmetic compositions 1030 formed from the ingredients 1014A, 1014B and 1014C. A third cluster 1035C comprises the filtered cosmetic compositions 1030 formed from the ingredients 1014C and 1014D, in proportions substantially equal to 80/20. A fourth cluster 1035D comprises the filtered cosmetic compositions 1030 formed from the ingredients 1014C and 1014D, in proportions substantially equal to 40/60.

The processing phase 1200 further comprises a step 1250 of selecting at least one cluster 1035 of filtered cosmetic compositions 1030, referred to as selected cluster 1035*. The selected cluster 1035* is for example selected at random.

According to a variant, during the selection step 1250, M clusters 1035 are selected, M being greater than or equal to two, advantageously equal to four.

For this, the M selected clusters 1035* are preferably selected such that, for each selected cluster 1035*, the ingredients 1014 comprised in the filtered cosmetic compositions 1030 of said selected cluster 1035* are only comprised in said selected cluster 1035*. In other words, the ingredients 1014 comprised in the filtered cosmetic compositions 1030 of said selected cluster 1035* are exclusive to said cluster 1035*. Otherwise expressed, the ingredients 1014 comprised in the filtered cosmetic compositions 1030 of said selected cluster 1035* are not comprised in the filtered cosmetic compositions 1030 of the other selected clusters 1035*.

In other words, two selected clusters 1035* does not comprise filtered cosmetic compositions 1030 having the same ingredient 1014.

In the example illustrated in FIG. 9, the first 1035A and second 1035B clusters can therefore not belong to the M selected clusters 1035*. Similarly, the third 1035B and fourth 1035D clusters can therefore not belong to the M selected clusters 1035*.

Thus, in the example in FIG. 9, the selected clusters 1035* are for example:
the first 1035A and the third 1035C clusters,
the first 1035A and the fourth 1035D clusters,
the second 1035B and the third 1035C clusters, or
the second 1035B and the fourth 1035D clusters.

Preferably, the selected clusters 1035* are the clusters 1035 further comprising the filtered cosmetic compositions 1030 for which each associated colorimetric parameter 1013 is closest to the at least one target colorimetric parameter 1013*.

The processing phase 1200 further comprises a step 1260 of determining the target cosmetic composition 1011* according to the filtered cosmetic compositions 1030 of the selected cluster 1035*.

Advantageously, when several clusters 1035 are selected, during the determination step 1260, a target cosmetic composition 1011* is determined for each selected cluster 1035.

The determination step 1260 preferably comprises for each ingredient 1014 of the filtered cosmetic compositions 1030 of the selected cluster 1035*, computing an average value of the quantities of said ingredient 1014 from said filtered cosmetic compositions 1030. In the example in FIG. 9, if the selected cluster 1035* is the first cluster 1035A, said computing is the computing of an average value of the quantity of ingredient 1014A in each filtered cosmetic composition 1030 of the first cluster 1035A and the computing of an average value of the quantity of ingredient 1014B in each filtered cosmetic composition 1030 of the first cluster 1035A.

The determination step 1260 further preferably comprises determining an average cosmetic composition 1040 comprising, for each of said ingredients 1014, a quantity equal to the respective computed average value of the ingredient 1014. In FIG. 9, the average cosmetic composition 1040A, 1040B, 1040C, 1040D of each cluster 1035A, 1035B, 1035C, 1035D is represented by a square.

It is then understood that the average cosmetic composition 1040 is a barycenter of the filtered cosmetic compositions 1030 of the selected cluster 1035*.

Advantageously, the determination step 1260 further comprises forming the target cosmetic composition 1011* according to the average cosmetic composition 1040.

For example, the target cosmetic composition 1011* is the average cosmetic composition 1040.

Alternatively, determining the target cosmetic composition 1011* according to the average cosmetic composition 1040 comprises computing at least one colorimetric parameter 1013 associated with the average cosmetic composition 1040, and optimizing the quantity of each ingredient 1014 of the average cosmetic composition 1040 such that the at least one colorimetric parameter 1013 associated with the average cosmetic composition 1040 approaches the at least one target colorimetric parameter 1013*.

In other words, an optimization algorithm is applied to a difference between the at least one colorimetric parameter 1013 associated with the average cosmetic composition 40 and the at least one target colorimetric parameter 1013*, to reduce this difference by varying the quantity of each ingredient 1014 of the average cosmetic composition 1040.

According to this variant, the target cosmetic composition 1011* is the average cosmetic composition 1040 resulting from optimizing the quantity of its ingredients 1014.

The computing of the at least one colorimetric parameter 1013 associated with the average cosmetic composition 1040 is for example implemented by the trained model.

Furthermore and optionally, when M clusters 1035 are selected, said determination step 1260 is repeated for each selected cluster 1035*. Thus, a target composition 1011* is determined for each selected cluster 1035*.

Furthermore and optionally, the processing phase 1200 further comprises a step 1270 of sending the or each target cosmetic composition 1011*, to the display screen 1016. The display screen 1016 then displays the or each target cosmetic composition 11*, intended for the operator of the determination device 1010.

According to another optional addition, the processing phase 1200 further comprises a step 1280 of producing at least one sample of the or each target cosmetic composition 1011*. The at least one sample is preferably produced by the unit 1017 for producing cosmetic compositions 1011.

Alternatively, the production step 1280 comprises receiving, from the operator, a selection of one or more target cosmetic compositions 1011* to be produced, from the target cosmetic compositions 1011* determined. The production unit 1017 then produces only the sample(s) of the target cosmetic compositions 1011* selected by the operator.

The sample(s) of target cosmetic compositions 1011* produced are intended to be applied on hair 1012, preferably a lock of hair 1012, to verify the colorimetric parameter(s).

Thus, following the application of each sample on hair 1012, the colorimetric parameters are measured using the same sensors used to form the training data 1018.

Alternatively, the processing phase 1200 does not comprise the sending step 1270 and/or the production step 1280.

Also alternatively, each cosmetic composition 1011 is capable of being applied to any type of hairs 1012, and not only hair, for example to eyelashes, eyebrows or a beard of the user.

The processing phase 1200 is preferably repeated a plurality of times, forming iterations. At each iteration, the at least one target colorimetric parameter 1013* acquired during the acquisition step 1210 is distinct from the at least one target colorimetric parameter 1013* acquired for the preceding iteration(s).

Preferably, during each repetition, i.e., during each successive iteration following the first iteration, the steps of obtaining 1220 the set of cosmetic compositions, and obtaining 1230 at least one colorimetric parameter 1013 associated with each cosmetic composition 1011 under test, and of formation 1240, are not implemented.

All the variants and optional additions listed above can be combined with each other.

With the determination method according to the second invention, it is possible to obtain automatically the target cosmetic composition 1011* using the at least one target colorimetric parameter 1013*.

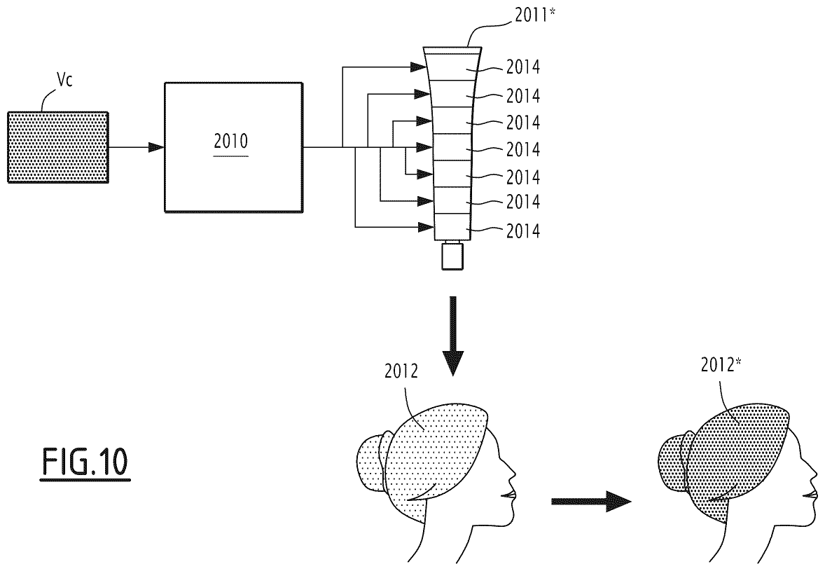

A third invention will now be described with reference to FIGS. 10 to 16.

The present third invention relates to a method for determining a target cosmetic composition for coloring hairs, particularly hair, according to at least one target colorimetric parameter. The present third invention also relates to an associated computer program product and electronic determination device.

The present third invention relates to the field of cosmetic products, preferably cosmetic compositions for coloring hairs, in particular hair.

More generally, a “cosmetic product” is a product as defined in Regulation EC No. 1223/2009 of the European Parliament and of the Council of Nov. 30, 2009, relating to cosmetic products.

An objective of the cosmetic industry is to improve the experience of its consumers. There is in particular a strong trend to propose products that are increasingly adapted to the needs and characteristics specific to the user. This trend is generally called “customization”.

Customizing cosmetic products and services can concern any part of the human body but is of particular interest for exposed body parts, such as the face (makeup or care products), and the hair or beard where applicable (coloring products for example).

In the field of cosmetic compositions for coloring hair(s), in particular hair, novel cosmetic compositions are regularly developed to adapt to user demand.

These novel cosmetic compositions are aimed, in a first case, at achieving a novel color or a novel visual effect of the hairs on which they are applied. In a second case, these novel cosmetic compositions are aimed at achieving a previously known color but comprise ingredients not previously used for this purpose. This second case is of particular interest when the procurement of certain ingredients becomes difficult, is accompanied by high costs, or poses a risk for the environment.

However, obtaining novel cosmetic compositions results from a lengthy and complex process.

Firstly, expert chemists develop cosmetic composition prototypes. These prototypes result from the experience of the expert chemists and are governed by certain chemical principles.

Then, these prototypes are tested individually in a laboratory to check that the visual effect obtained meets the expert chemists' expectations.

In some cases and unpredictably, the visual effect associated with a newly developed prototype does not meet expectations.

It is then known to abandon the prototype to test a new prototype which does not necessarily have a link with the first.

In this case, no capitalization is made on the prototype failing to meet expectations. There is therefore a need for tools making it possible to benefit from prototypes failing to meet expectations.

To this end, the present third invention relates to a method for adapting an initial cosmetic composition, intended for coloring hairs, in particular hair, so that the value of at least one colorimetric parameter of the adapted cosmetic composition corresponds to a target value, the method being implemented by an electronic adaptation device and comprising a processing phase including the following steps:
obtaining a target value for the at least one colorimetric parameter,
obtaining data relating to an initial cosmetic composition including a plurality of ingredients and for each ingredient, an associated quantity,
obtaining an initial value of the at least one colorimetric parameter for the initial cosmetic composition, the initial value resulting from a measurement following the application of the initial cosmetic composition on hairs, the initial value differing from the target value by a first deviation,
adapting the initial cosmetic composition to compensate for the first deviation between the initial value and the target value, the adaptation comprising:
   varying the quantity of each ingredient in the initial cosmetic composition, and
   evaluating, using an automatic determination model, the influence of each variation on the value of the at least one colorimetric parameter, the automatic determination model having been previously trained to determine a value of at least one colorimetric parameter according to a cosmetic composition,
      the adapted cosmetic composition being obtained following the adaptation.

Thanks to the adaptation step, it is possible to obtain, from an initial composition, an adapted cosmetic composition of which the value of the at least one colorimetric parameter approaches the target value, in particular via the automatic determination model. It is then possible to capitalize on prior research resulting in a prototype of which the at least one colorimetric parameter did not has a value equal to the target value.

According to particular embodiments, the adaptation method according to the third invention comprises one or several of the following characteristics, taken in isolation or in any technically possible combination:
the adapted cosmetic composition has an adapted value for the at least one colorimetric parameter, the adapted value resulting from a measurement following the application of the adapted cosmetic composition on hairs, the adapted cosmetic composition being such that a second deviation between the adapted value and the target value is less than the first deviation;
the processing phase further comprises the following step:
   computing, using the automatic determination model, a matrix, referred to as Jacobian matrix, representing a variation of the value of the at least one colorimetric parameter of the initial cosmetic composition according to a variation of the quantity of each ingredient of the initial cosmetic composition, during the evaluation of the influence of each variation on the value of the at least one colorimetric parameter of the adaptation step of the processing phase, said influence being evaluated by computing a value of a cost function dependent on the Jacobian matrix and each variation;
the cost function complies with the following formula:

$$FC = ‖ Vi - Vc + J Δ mp ‖$$

where FC is the cost function,
Vi is the initial value of the at least one colorimetric parameter,
Vc is the target value,
J is the Jacobian matrix, and
Δmp is a vector comprising each ingredient quantity variation;
during the adaptation step of the processing phase, the variation and the evaluation are repeated several times by an optimization algorithm;
during the adaptation step of the processing phase, the optimization algorithm applied is an optimization algorithm constrained by a set of viability constraint(s) of the cosmetic composition(s),
the initial cosmetic composition and the adapted cosmetic composition complying with the set of viability constraint(s);
the method comprises, prior to the processing phase, a training phase including the following steps:
   receiving a set of viability constraint(s) of cosmetic composition(s),
   acquiring a set of training data, each training data item being specific for a cosmetic composition complying with the set of viability constraint(s) received,
   each training data item comprising:
      a set of magnitudes representing the quantity of each ingredient of the cosmetic composition, and
      a value of the at least one colorimetric parameter associated with the cosmetic composition, and
   training an artificial intelligence model based on the set of training data, to obtain a trained model capable of determining, from a cosmetic composition, a value of the at least one colorimetric parameter of the cosmetic composition,
during the adaptation step of the processing phase, the model for determining at least one cosmetic composition colorimetric parameter being the model trained during the training step of the training phase;
the processing phase further comprises a step of producing a sample of the adapted cosmetic composition, in order to measure the adapted value of the at least one colorimetric parameter of the adapted colorimetric composition; and
the processing phase is repeated at least one forming at least two iterations, during the steps of obtaining the or each repetition of the processing phase, the initial cosmetic composition being the adapted cosmetic composition obtained in the preceding iteration and the initial value resulting from a measurement following the application of the adapted cosmetic composition from the preceding iteration on hairs.

The present third invention also relates to a computer program product on which a computer program comprising program instructions is stored, the computer program being loaded onto a data processing unit and implementing such a method when the computer program is implemented on the data processing unit.

The present third invention also relates to an electronic device for adapting an initial cosmetic composition, intended for coloring hairs, in particular hair, so that the value of at least one colorimetric parameter of the adapted cosmetic composition corresponds to a target value, the electronic adaptation device being capable of implementing such a determination method.

The present third invention also relates to a readable information medium on which a computer program product comprising program instructions is stored, the computer program being loaded onto a data processing unit and implementing such a determination method when the computer program is implemented on the data processing unit.

Other features and advantages of the third invention will appear when reading the following description of embodiments of the third invention, provided solely as an example and with reference to the drawings which are:

FIG. 10 is a schematic view of a concept of the third invention;

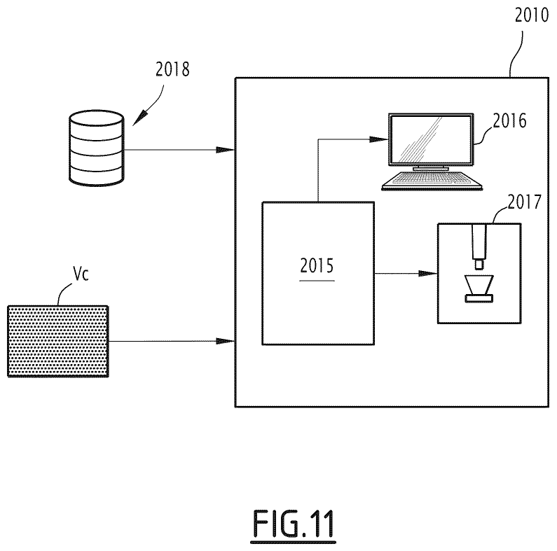

FIG. 11 is a schematic view of an electronic adaptation device according to the third invention;

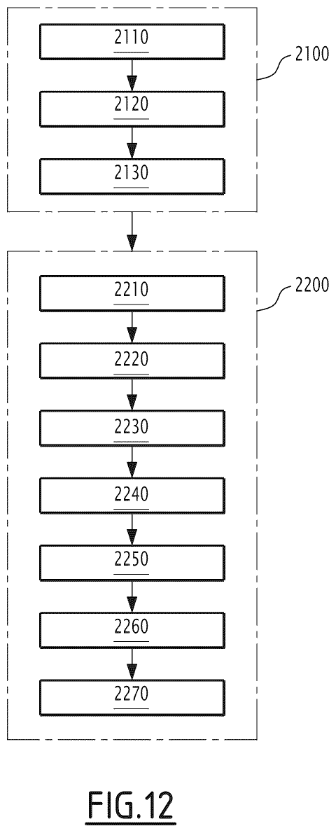

FIG. 12 is a flow chart of an adaptation method according to the third invention implemented by the electronic adaptation device of FIG. 11;

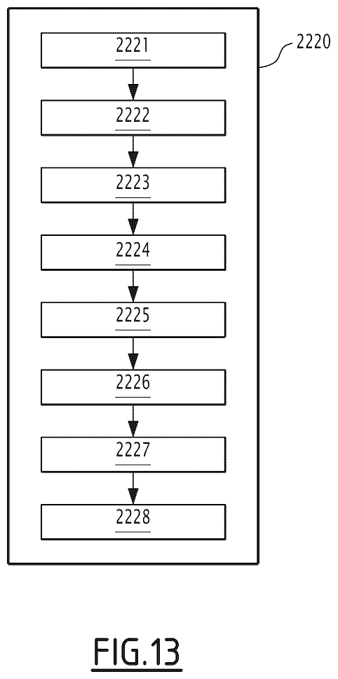

FIG. 13 is a flow chart of a step of the adaptation method of FIG. 12;

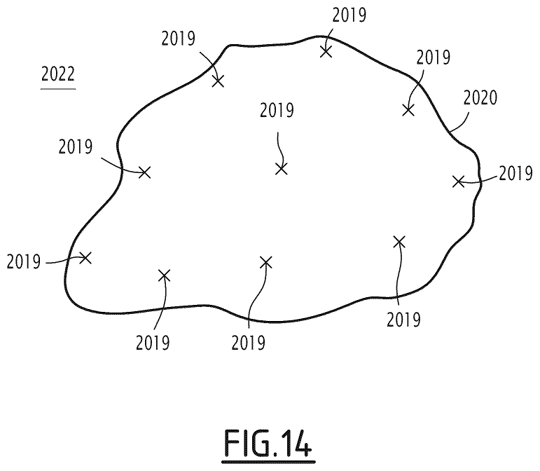

FIG. 14 is a two-dimensional schematic representation of a reduced viable cosmetic composition space;

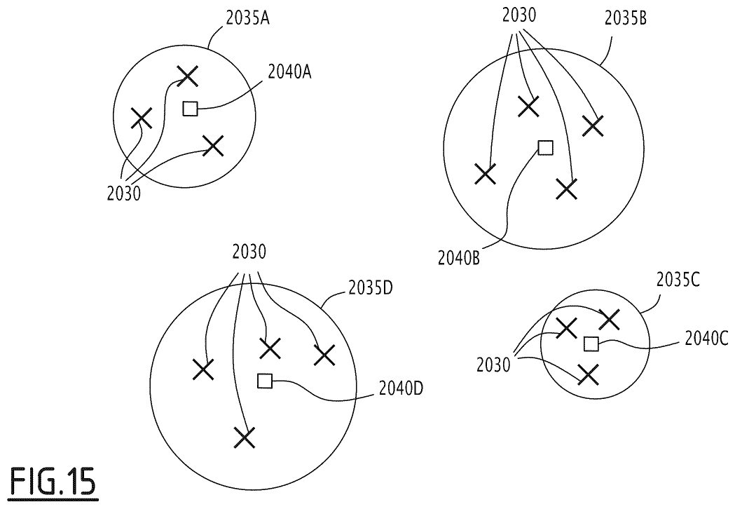

FIG. 15 is a two-dimensional schematic representation of a sub-step illustrated in FIG. 13 of the adaptation method illustrated in FIG. 12; and

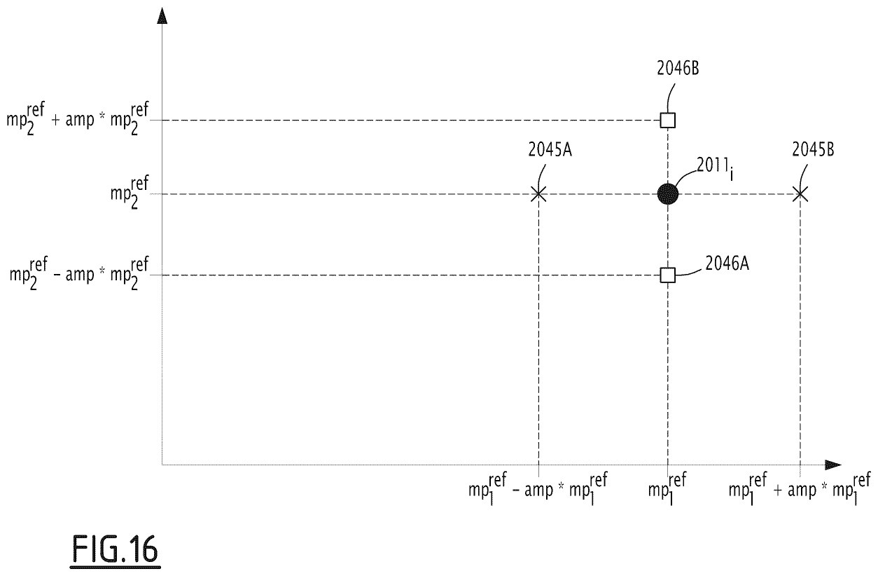

FIG. 16 is a schematic representation of a step of the adaptation method illustrated in FIG. 12.

In FIG. 10, an electronic device 2010 for adapting an initial cosmetic composition 2011ᵢ, intended for coloring hairs 2012, in particular hair, so that the value of at least one colorimetric parameter of the adapted cosmetic composition 2011* corresponds to a target value Vc, is represented. The verb “correspond” means that the value of the at least one colorimetric parameter approaches the target value Vc in relation to an initial value described hereinafter.

The initial cosmetic composition 2011ᵢ is not represented in FIG. 10 because, in an embodiment, the adaptation device 2010 is capable of determining it itself, as will be explained hereinafter.

As seen in FIG. 10, the adapted cosmetic composition 2011* is a cosmetic composition for coloring hairs 2012. Preferably, the target cosmetic composition 2011* is capable of being applied on the hair 2012 of a user, to color said hair 2012.

The adapted cosmetic composition 2011* comprises a plurality of ingredients 2014 capable of interacting with each other, and with the hair 2012 of the user, to color it.

Preferably, during this interaction, the ingredients 2014 of the adapted cosmetic composition 2011* start by decolorizing the hair 2012. Then, the ingredients 2014 of the adapted cosmetic composition 2011* interact with the decolorized hair to fix pigments of the chosen color and thus form colored hair 2012*.

The colored hair 2012* has a color corresponding to the target value Vc of the at least one colorimetric parameter.

The ingredients 2014 are for example present in the adapted cosmetic composition 2011* in the form of powder, gel, emulsion or oil.

The electronic adaptation device 2010 is configured to adapt a quantity of each ingredient 2014 in the initial cosmetic composition 2011ᵢ, thus forming the adapted cosmetic composition 2011*. The adapted cosmetic composition 2011* is intended to produce a hair coloring corresponding to the at least target colorimetric parameter Vc, when it is applied on the user's hair 2012.

With reference to FIG. 11, the adaptation device 2010 comprises a processing unit 2015.

The adaptation device 2010 optionally further comprises a display screen 2016 and/or a unit 2017 for producing cosmetic compositions 2011.

The processing unit 2015 comprises, for example, a calculator interacting with a computer program product. For example, the processing unit 2015 is a computer.

The computer comprises, for example, a processor comprising a data processing unit, memories and an information medium reader, as well as optionally a human-machine interface.

The computer program product includes an information medium.

The information medium is a computer-readable medium, usually by the data processing unit. The readable data medium is a medium adapted to store electronic instructions and capable of being coupled with a computer system bus.

For example, the information medium is a USB flash disk, a floppy disk or flexible disk (“floppy disk”), an optical disk, a CD-ROM, a magnetic-optical disk, a ROM memory, a RAM memory, an EPROM memory, an EEPROM memory, a magnetic card or an optical card.

The computer program comprising program instructions is stored on the information medium.

The computer program can be loaded on the processing unit 2015 and is adapted to implement a method for adapting an initial cosmetic composition 2011ᵢ to obtain an adapted cosmetic composition 2011*, when the computer program is implemented on the processing unit of the computer. Such a determination method will be described hereinafter in the description.

The operation of the electronic device 2010, implementing a method for adapting the initial cosmetic composition 2011ᵢ, will now be described with reference to the flow charts of FIGS. 12 and 13, as well as the examples of FIGS. 14 to 16.

The determination method optionally comprises a training phase 2100.

The training phase 2100 comprises a step 2110 of receiving a set E_{cv} of viability constraint(s) of cosmetic composition(s) 2011.

Preferably, each cosmetic composition(s) 2011 comprises at least one ingredient 2014 of a first type and at least one ingredient 2014 of a second type.

Each ingredient 2014 of the first type is for example a base, and each ingredient 2014 of the second type is for example a coupler.

Preferably, each ingredient 2014 is chosen from a predefined list of ingredients. Preferably, the set E_{cv} of viability constraint(s) of cosmetic composition(s) 2011 comprises one or more of the following constraints:
a ratio between a quantity of ingredient 2014 of the first type and a quantity of ingredient 2014 of the second type is between a first threshold and a second threshold,
a total quantity of the cosmetic composition 2011 is less than a third threshold,
a quantity of each ingredient 2014 in the cosmetic composition 2011 is less than a fourth threshold, and
a number of ingredients 2014 in the cosmetic composition 2011 is less than a fifth threshold.

Advantageously, the set of constraint(s) E_{cv} comprises each of the constraints cited above.

These constraints are optionally derived from chemical principles ensuring the viability of the cosmetic composition 2011. The expression “viability of the cosmetic composition 2011” is understood here to mean a composition complying with the set of constraint(s): is effective, and/or poses no risks for the user, and/or complies with production standards, and is preferably environmentally friendly.

The optional training phase 2100 further comprises a step 2120 of acquiring a set of training data 2018.

Each training data item 2018 of the set of training data 2018 is specific to a cosmetic composition 2011 complying with the set of viability constraint(s) received during the receiving step 2110.

Each training data item 2018 comprises a set of magnitudes representing the quantity of each ingredient 2014 in the cosmetic composition 2011, and a value of at least one colorimetric parameter associated with the cosmetic composition 2011.

Each magnitude is for example a quantity of the corresponding ingredient 2014 in the associated cosmetic composition 2011.

Each quantity of ingredient 2014 is optionally an ingredient mass, an ingredient volume, a mass percentage of the ingredient 2014 in the cosmetic composition 2011, or a volume percentage of the ingredient 2014 in the cosmetic composition 2011.

The or each colorimetric parameter characterizes the cosmetic composition 2011 for coloring hair 2012.

Preferably, each colorimetric parameter is representative of a visual effect of the hair 2012 on which the cosmetic composition 2011 is applied.

Advantageously, the at least one colorimetric parameter comprises:
a triplet of values characterizing a hair 2012 color after applying the cosmetic composition 2011, and/or
a color fade value of the cosmetic composition 2011 after washing the hair 2012, and/or
a selectivity value characterizing a difference in color between a root and a tip of the hair 2012.

Preferably, each training data item 2018 comprises each of the values cited above.

The triplet of values is for example is the CIE L*a*b* chromatic space value triplet.

The CIE L*a*b* color space, often abbreviated as CIELAB, is a color space for surface colors defined by the International Commission on Illumination (CIE) in 1976. It is based on evaluations of the CIE XYZ system, and abandons linearity to more accurately show up differences between colors perceived by the human eye. Three magnitudes characterize colors in this model, namely the lightness L* derived from the luminance (Y) of the XYZ evaluation, and two parameters a* and b* that express the color difference from the color of a gray surface with the same lightness, as the chrominance. The definition of a gray, uncolored, achromatic surface implies that the composition of the light that illuminates the colored surface is explicitly indicated. This illuminant is often daylight corresponding to the D65 normalized standard.

Also, in this case, the triplet of values comprises a lightness value L*, and two color difference values a*, b* from the color of a gray surface with the same lightness.

Any other color space could be envisaged.

Alternatively, the triplet of values is an RGB triplet. The triplet then comprises a value R for the red color, a value G for green and a value B for blue.

More generally, any representations making it possible to associate spectra could also be used in this context.

Indeed, the color of a hair is not identical at all points of the hair. In particular, the tips of hair 2012 are generally lighter than the roots. Thus, the selectivity value is parameter of interest.

Preferably, during the acquisition step 2120 of the training phase 2100, the set of training data 2018 is acquired, at least in part, via a sensor capable of measuring the value of the at least one colorimetric parameter.

The sensor is for example a spectrocolorimeter.

According to an embodiment, the sensor is capable of capturing images of zones of hairs of the individual and extracting the colorimetric measurements from the images captured.

Preferably, each training data item 2018 is derived from a viable cosmetic composition 2011, i.e., complying with the set E_{cv} of viability constraint(s).

Preferably, each training data item corresponds to a marketed cosmetic composition. Thus, before the marketing of said cosmetic compositions, the value of their colorimetric parameters has been measured during testing on hair 2012.

Furthermore and optionally, the cosmetic compositions associated with the training data are preselected by expert chemists from all the marketed cosmetic compositions.

The optional training phase 2100 further comprises a step 2130 of training an artificial intelligence model using the set of training data 2018, to obtain a trained model.

Preferably, the artificial intelligence model comprises at least one of the following models: a support vector machine, a random forest, a gradient boosting mechanism, or kriging, also known as Gaussian process regressor.

In each of the above cases, the artificial intelligence model comprises adjustable parameters. During the training step 2130, each adjustable parameter is adjusted such that, when a respective set of magnitudes of a training data item 2018 is given as an input, said trained model supplies as an output, a value of the at least one colorimetric parameter substantially identical to the value of the at least one colorimetric parameter of said training data item 2018.

It is clear that the aim of the training phase 2100 described above is to obtain the trained model.

According to a variant not shown, the determination method does not comprise the training phase 2100.

For example, the method comprises, instead, a step of receiving the trained model. The method further comprises a processing phase 2200.

The processing phase 2200 comprises a step 2210 of obtaining the target value Vc for the at least one colorimetric parameter. The target value Vc of the at least one colorimetric parameter is for example supplied by an operator of the determination device 2010.

The processing phase comprises a step 2220 of obtaining data relating to the initial cosmetic composition 2011ᵢ including several ingredients 2014 and for each ingredient 2014, an associated quantity. Hereinafter in the description, the expressions “obtaining the initial cosmetic composition 2011ᵢ” and “obtaining data relating to the initial cosmetic composition 2011ᵢ” are equivalent.

With reference to FIG. 13, the step 2220 of obtaining the initial cosmetic composition 2011ᵢ comprises a sub-step 2221 of obtaining a set of cosmetic compositions 2011 under test.

The set of cosmetic compositions 2011 under test is preferably formed from several test data items 2019.

The test data item 2019, for each cosmetic composition 2011 under test, a set of magnitudes representing the quantity of each ingredient 2014 in the cosmetic composition 2011 under test.

Preferably, the ingredients 2014 of each cosmetic composition 2011 under test are chosen from the predefined list of ingredients 2014 described above.

Each cosmetic composition 2011 under test complies with the set E_{cv} of viability constraint(s) received during the training phase 2100.

If the method does not comprise the training phase 2100, then the sub-step 2221 of obtaining the set of cosmetic compositions 2011 under test further comprises receiving said set of viability constraint(s).

The sub-step 2221 of obtaining the set of cosmetic compositions 2011 under test advantageously comprises receiving a number N corresponding to a number of cosmetic compositions 2011 under test. Optionally, the number N is greater than or equal to two. Preferably, the number N is greater than one hundred, for example equal to one hundred and fifty.

The obtaining sub-step 2221 furthermore advantageously comprises generating N test data items 2019. Each test data item 2019 relates to a cosmetic composition 2011 of which the ingredients 2014 are chosen from the predefined list of ingredients 2014 and such that the cosmetic composition 2011 under test associated with each test data item 2019 is within a reduced viable cosmetic composition space 2020. The reduced viable cosmetic composition space 2020 only comprises cosmetic compositions 2011 complying with the set E_{cv} of viability constraint(s). The test data items 2019 are representative of the reduced viable cosmetic composition space 2020.

The expression “test data items 2019 representative of the reduced space 2020” is understood here to mean that the test data items 2019 are aptly chosen in the reduced space 2020 to substantially cover the entire reduced space 2020.

FIG. 14 illustrates, via a two-dimensional block diagram, a set 2022 representing all the possible cosmetic compositions 2011 and the reduced space 2020. It is clear that this set 2022 and this space 2020 cannot, in fact, be represented two-dimensionally but rather in a number of dimensions equal to the number of ingredients 2014 in the predefined list of ingredients.

In FIG. 14, the set 2022 of all the possible cosmetic compositions 2011 corresponds to the map in FIG. 14. As seen in FIG. 14, the reduced space 2020 is included in said set 2022 because the reduced space 2020 only comprises the cosmetic compositions 2011 of the set 2020 complying with the set E_{cv} of viability constraint(s).

Again in FIG. 14, the test data items 2019 in the reduced space 2020 can be seen. For example, ten test data items 2019 are represented in FIG. 14.

Preferably, generating N test data items 2019 comprises computing magnitudes of the N test data items 2019 such that, for each test data item 2019, the distance between said test data item 2019 and the other test data items 2019 is maximum.

Each magnitude of each test data item 2019 represents the quantity of an ingredient 2014 in the corresponding cosmetic composition 2011 under test.

Generating N test data items 2019 comprising computing magnitudes of N sets of magnitudes for which a distance between each pair of sets of magnitudes is maximum. Each magnitude of a respective set of magnitudes represents the quantity of an ingredient 2014 in a cosmetic composition 2011 associated with said set of magnitudes, said cosmetic composition 2011 complying with the set of constraint(s). For example, a test data item 2019 comprises a first magnitude equal to 4 mol of the first ingredient 2014A and 6 mol of a second ingredient 2014B.

Advantageously, each test data item 2019 is a vector comprising a coefficient for each ingredient 2014 of the predefined list of ingredients.

Each coefficient represents the quantity of said ingredient 2014 in the respective cosmetic composition 2011 of the associated vector.

A distance between two test data items 2019, i.e., between two vectors, is for example defined by the algebraic norm according to the following equation:

$$distance ( X , Y ) = ‖ X - Y ‖ = \sqrt{{\sum }_{i = 1}^{P} {( {x}_{i} - {y}_{i} )}^{2}} \qquad [ MATH 1 ]$$

where X and Y are two sets of P magnitudes,
x_{i,i=1, . . . ,P} and y_{i,i=1, . . . ,P} are the respective magnitudes of said sets X and Y,
√ is the square root function, and
Σ Is the sum operator.

The N vectors are computed for example by applying a design of experimentation technique, also known as DoE technique. Advantageously, the DoE technique implements an SFD (space filling design) algorithm. This algorithm makes it possible, even with a reduced number of vectors, to ensure that the vectors chosen are representative of the reduced space 2020. Indeed, when the number of vectors is low, the hypotheses of the law of large numbers are not sufficiently fulfilled for a random distribution of vectors to be able to be representative of the reduced space 2020.

In FIG. 14, it can be seen that the test data items 2019 are spaced apart from one another so as to maximize the distances between the test data items 2019.

Alternatively, the obtaining sub-step 2221 only comprises acquiring one or more test data items 2019 chosen by an operator of the adaptation device 2010. In this case, the operator chooses one or more cosmetic compositions by selecting, for each cosmetic composition 2011, ingredients 2014 from the predefined list of ingredients, and the quantity of each of said ingredients 2014. When choosing, the operator ensures that each cosmetic composition 2011 chosen complies with the set of constraint(s).

The set of cosmetic compositions under test is formed by the N test data items 2019. The step 2220 of obtaining the initial cosmetic composition further comprises a sub-step 2222 of obtaining a value of at least one colorimetric parameter associated with each cosmetic composition 2011 under test.

Preferably, the obtaining sub-step 2222 comprises applying, to the test data items 2019, forming the set of cosmetic compositions 2011 obtained, the trained model to determine the value of the at least one colorimetric parameter of the or each cosmetic composition 2011 under test.

Preferably, during the obtaining sub-step 2222, each test data item 2019 is supplied successively to the trained model. The trained model then determines, for each test data item 2019, the value of the associated colorimetric parameter(s).

Advantageously, during the obtaining sub-step 2222, the cosmetic composition 2011 and the value of the at least one colorimetric parameter associated with each test data item are stored in the memory or memories of the processing unit 2015.

Alternatively, the obtaining sub-step 2222 comprises receiving, from the operator of said device 2010, the value of the at least one colorimetric parameter associated with each cosmetic composition 2011 of the set of cosmetic compositions 2011 under test.

In this case, the value of the at least one colorimetric parameter is for example measured by the operator of the determination device 2010 for example by means of a sensor, as explained above for the test data items 2019.

This variant is combined particularly advantageously with the variant of the sub-step 2221 of obtaining the set of cosmetic compositions 2011 under test, whereby the test data items 2019 associated with the cosmetic compositions 2011 under test are received from the operator.

The step 2220 of obtaining the cosmetic composition further comprises a filtering sub-step 2223, during which the cosmetic compositions 2011 under test are filtered according to the value of the at least one colorimetric parameter associated with them. The filtering sub-step 2223 makes it possible to obtain filtered cosmetic compositions 2030, for which the value of the at least one colorimetric parameter meets a criterion relating to the target value Vc.

Preferably, the criterion is that a relative difference between the value of the at least one colorimetric parameter associated with each cosmetic composition 2011 under test and the target value Vc, is less than a sixth threshold. The filtered cosmetic compositions 2030 are only the cosmetic compositions 2011 under test for which said relative difference is less than the sixth threshold. In other words, the filtered cosmetic compositions 2030 are the cosmetic compositions 2011 under test for which the value of each colorimetric parameter deviates, from the target value Vc, by not more than the sixth threshold.

The step 2220 of obtaining the initial cosmetic composition 2011ᵢ further comprises a sub-step 2224 of forming several clusters 2035 of filtered cosmetic compositions 2030. For each cluster 2035, the filtered cosmetic compositions 2030 comprise the same ingredients 2014. The formation sub-step 2224 comprises for example the application of an unsupervised learning algorithm, such as a k-means algorithm.

The filtered cosmetic compositions 2030 of each cluster 2035 are substantially similar. In other words, two filtered cosmetic compositions 2030 comprising the same ingredients 2014 but in substantially different proportions are not comprised in the same cluster 2035. For example, a filtered cosmetic composition 2030 comprising 10% of an ingredient 2014A and 90% of an ingredient 2014B is not comprised in the same cluster 2035 as a filtered cosmetic composition 2030 comprises 80% of the ingredient 2014A and 20% of the ingredient 2014B.

In FIG. 15, several clusters 2035A, 2035B, 2035C, 2035D are represented by ellipses. The filtered cosmetic compositions 2030 of each cluster 2035A, 2035B, 2035C, 2035D are represented by crosses.

As seen in FIG. 15, a first cluster 2035A comprises the filtered cosmetic compositions 2030 formed from the ingredients 2014A and 2014B. A second cluster 2035B comprises the filtered cosmetic compositions 2030 formed from the ingredients 2014A, 2014B and 2014C. A third cluster 2035C comprises the filtered cosmetic compositions 2030 formed from the ingredients 2014C and 2014D, in proportions substantially equal to 80/20. A fourth cluster 2035D comprises the filtered cosmetic compositions 2030 formed from the ingredients 2014C and 2014D, in proportions substantially equal to 40/60.

The step 2220 of obtaining the initial cosmetic composition 2011ᵢ further comprises a sub-step 2225 of selecting at least one cluster 2035 of filtered cosmetic compositions 2030, referred to as selected cluster 2035*. The selected cluster 2035* is for example selected at random.

According to a variant, during the selection sub-step 2225, M clusters 2035 are selected, M being greater than or equal to two, advantageously equal to four.

For this, the M selected clusters 2035* are preferably selected such that, for each selected cluster 2035*, the ingredients 2014 comprised in the filtered cosmetic compositions 2030 of said selected cluster 2035* are only comprised in said selected cluster 2035*. In other words, the ingredients 2014 comprised in the filtered cosmetic compositions 2030 of said selected cluster 2035* are exclusive to said cluster 2035*. Otherwise expressed, the ingredients 2014 comprised in the filtered cosmetic compositions 2030 of said selected cluster 2035* are not comprised in the filtered cosmetic compositions 2030 of the other selected clusters 2035*

In other words, two selected clusters 2035* does not comprise filtered cosmetic compositions 2030 having the same ingredient 2014.

In the example illustrated in FIG. 14, the first 2035A and second 2035B clusters can therefore not belong to the M selected clusters 2035*. Similarly, the third 2035B and fourth 2035D clusters can therefore not belong to the M selected clusters 2035*.

Thus, in the example in FIG. 15, the selected clusters 2035* are for example:
the first 2035A and the third 2035C clusters,
the first 2035A and the fourth 2035D clusters,
the second 2035B and the third 2035C clusters, or
the second 2035B and the fourth 2035D clusters.

Preferably, the selected clusters 2035* are the clusters 2035 further comprising the filtered cosmetic compositions 2030 for which the value of each associated colorimetric parameter is closest to the target value Vc.

The step 2220 of obtaining the initial cosmetic composition further comprises a step 2226 of determining a potential cosmetic composition 2011ₚ according to the filtered cosmetic compositions 2030 of the selected cluster 2035*

Advantageously, when several clusters 2035 are selected, during the determination sub-step 2226, a potential cosmetic composition 2011ₚ is determined for each selected cluster 2035.

The determination sub-step 2226 preferably comprises for each ingredient 2014 of the filtered cosmetic compositions 2030 of the selected cluster 2035*, computing an average value of the quantities of said ingredient 2014 from said filtered cosmetic compositions 2030. In the example in FIG. 15, if the selected cluster 2035* is the first cluster 2035A, said computing is the computing of an average value of the quantity of ingredient 2014A in each filtered cosmetic composition 2030 of the first cluster 2035A and the computing of an average value of the quantity of ingredient 2014B in each filtered cosmetic composition 2030 of the first cluster 2035A.

The determination sub-step 2226 further preferably comprises determining an average cosmetic composition 2040 comprising, for each of said ingredients 2014, a quantity equal to the respective computed average value of the ingredient 2014. In FIG. 15, the average cosmetic composition 2040A, 2040B, 2040C, 2040D of each cluster 2035A, 2035B, 2035C, 2035D is represented by a square.

It is then understood that the average cosmetic composition 2040 is a barycenter of the filtered cosmetic compositions 2030 of the selected cluster 2035*.

Advantageously, the determination sub-step 2226 further comprises forming the potential cosmetic composition 2011ₚ according to the average cosmetic composition 2040.

For example, the potential cosmetic composition 2011ₚ is the average cosmetic composition 2040.

Alternatively, determining the potential cosmetic composition 2011ₚ according to the average cosmetic composition 2040 comprises computing the value of at least one colorimetric parameter associated with the average cosmetic composition 2040, and optimizing the quantity of each ingredient 2014 of the average cosmetic composition 2040 such that the value of the at least one colorimetric parameter associated with the average cosmetic composition 2040 approaches the target value Vc.

In other words, a first optimization algorithm is applied to a difference between the value of the at least one colorimetric parameter associated with the average cosmetic composition 2040 and the target value Vc, to reduce this difference by varying the quantity of each ingredient 2014 of the average cosmetic composition 2040.

According to this variant, the potential cosmetic composition 2011ₚ is the average cosmetic composition 2040 resulting from optimizing the quantity of its ingredients 2014.

The computing of the at least one colorimetric parameter associated with the average cosmetic composition 2040 is for example implemented by the trained model.

Furthermore and optionally, when M clusters 2035 are selected, said determination sub-step 2226 is repeated for each Thus, a potential composition 2011ₚ is determined for each selected cluster 2035*.

Furthermore and optionally, the step 2220 of obtaining the initial cosmetic composition further comprises a sub-step 2227 of sending the or each potential cosmetic composition 2011ₚ, to the display screen 2016. The display screen 2016 then displays the or each potential cosmetic composition 2011ₚ, intended for the operator of the determination device 2010.

According to another optional addition, the obtaining step 2220 further comprises a sub-step 2228 of producing at least one sample of the or each potential cosmetic composition 2011ₚ. The at least one sample is preferably produced by the unit 2017 for producing cosmetic compositions 2011.

The or each potential cosmetic composition 2011ₚ, of which a sample is produced, is then the initial cosmetic composition 2011ᵢ.

Advantageously, the production sub-step 2228 comprises receiving, from the operator, a selection of a potential cosmetic composition 2011ₚ to be produced, from the potential cosmetic compositions 2011ₚ determined. The production unit 2017 then produces only the sample of the potential cosmetic compositions 2011ₚ selected by the operator.

In this case, only the potential cosmetic composition 2011ₚ, of which a sample is produced, is the initial cosmetic composition 2011ᵢ.

It is then understood that the at least one colorimetric parameter of the initial cosmetic composition 2011ᵢ has, according to the trained model, a value substantially equal to the target value Vc.

The initial cosmetic composition 2011ᵢ is intended to be applied on hair 2012, preferably a lock of hair 2012, to verify the value of the at least one colorimetric parameter. Thus, following the application of the initial cosmetic composition 2011ᵢ on hair 2012, the value of the at least one colorimetric parameter is measured using the same sensors used to form the training data 2018, forming an initial value Vi of the at least one colorimetric parameter.

As stated above, it is then understood that the adaptation device 2010 does not receive the initial cosmetic composition 2011ᵢ, but prepares it itself.

Alternatively, the step 2220 of obtaining the initial cosmetic composition 2011ᵢ does not comprise any of the sub-steps described above. Instead, during the obtaining step 2220, the adaptation device 2010 receives the initial cosmetic composition 2011ᵢ from the operator of the adaptation device 2010.

In this case, the operator ensures that the initial cosmetic composition 2011ᵢ complies with the set E_{cv} of viability constraints. The operator furthermore ensures that the at least one colorimetric parameter associated with said initial cosmetic composition 2011ᵢ has a value substantially equal to the target value Vc according to the trained model. Preferably, in this case, the order of the steps 2210 of obtaining the target value and of obtaining the initial cosmetic composition 2220 is reversed, the target value Vc being determined by applying the trained model to the initial cosmetic composition 2011ᵢ.

According to this variant, the initial cosmetic composition 2011ᵢ is nonetheless applied on hair 2012 and the value of the at least one associated colorimetric parameter is measured using the same sensors used to form the training data 2018, forming the initial value Vi of the at least one colorimetric parameter.

In some cases, the trained model is not perfect and the initial value Vi resulting from a measurement, does not correspond perfectly to the target value Vc.

The processing phase 2200 comprises a step 2230 of obtaining the initial value Vi, the initial value Vi resulting from the measurement following the application of the initial cosmetic composition 2011ᵢ on hairs 2012. The initial value Vi differs from the target value by a first deviation.

Optionally, the processing phase 2200 comprises a step 2240 of computing, using an automatic determination model of a value of at least one colorimetric parameter using a cosmetic composition, a matrix, referred to a Jacobian matrix J. The automatic determination model is preferably the trained model. The Jacobian matrix J represents a variation of the value V of the at least one colorimetric parameter of the initial cosmetic composition 2011ᵢ according to a variation of the quantity of each ingredient 2014 of the initial cosmetic composition 2011ᵢ.

The Jacobian matrix J is preferably computed for an operating point of the trained model equal to the initial cosmetic composition 2011ᵢ.

Optionally, a relative amplitude amp of computing the Jacobian matrix J is predefined. For each ingredient 2014 of the initial cosmetic composition 2011ᵢ, a pair of computing cosmetic compositions 2045A, 2045B, 2046A, 2046B is computed. For each pair, a first computing cosmetic composition 2045A, 2046A comprises the same quantity of each ingredient 2014 as the initial cosmetic composition 2011ᵢ except for one of the ingredients 2014 for which its quantity is reduced by the product of the relative amplitude amp and the quantity of said ingredient 2014 in the initial cosmetic composition 2011ᵢ. In other words, the quantity of said ingredient 2014 in the first computing composition 2045A, 2046A complies with the following equation:

$$m {p}_{i} = m {p}_{i}^{ref} - a m p * m {p}_{i}^{ref} = m {p}_{i}^{ref} ( 1 - amp )$$

where mpᵢ is the quantity of the i-th ingredient in the first computing cosmetic composition 2045A, 2046A, and

$$m {p}_{i}^{ref}$$

is the quantity of the i-th ingredient in the initial composition 2011ᵢ.

For the same pair, a second computing cosmetic composition 2045B, 2046B comprises the same quantity of each ingredient 2014 as the initial cosmetic composition 2011ᵢ except for said ingredient 2014 for which its quantity is increased by the product of the relative amplitude amp and the quantity of said ingredient 2014 in the initial cosmetic composition 2011ᵢ. In other words, the quantity of said ingredient in the first computing composition 2045B, 2046B complies with the following equation:

$$m {p}_{i} = m {p}_{i}^{ref} + a m p * m {p}_{i}^{ref} = m {p}_{i}^{ref} ( 1 + amp ) \qquad [ MATH 3 ]$$

where mpᵢ is the quantity of the i-th ingredient in the second computing cosmetic composition.

In FIG. 16, this computing is illustrated in a simple example for which the initial cosmetic composition 2011ᵢ only comprises two ingredients 2014. FIG. 16 represents a frame of reference wherein the quantity of a first ingredient is noted on the x-axis and the quantity of second ingredient is noted on the y-axis.

In FIG. 16, the initial cosmetic composition 2011ᵢ is represented by a circle. The first 2045A and second 2045B computing cosmetic compositions associated with the first ingredient are represented by crosses. The first 2046A and second 2046B computing cosmetic compositions associated with the second ingredient are represented by squares.

It is noted in FIG. 16 that, for each of the first 2045A and second 2045B computing cosmetic compositions associated with the first ingredient, the quantity of the second ingredient is equal to the quantity of the second ingredient in the initial composition 2011ᵢ. Similarly, for each of the first 2046A and second 2046B computing cosmetic compositions associated with the second ingredient, the quantity of the first ingredient is equal to the quantity of the first ingredient in the initial composition.

For each ingredient 2014, a first computing value VA of the at least one colorimetric parameter is determined by applying the automatic determination model to the first computing cosmetic composition 2045A, 2046A, and a second computing value VB of the at least one colorimetric parameter is determined by applying the automatic determination model to the second computing cosmetic composition 2045B, 2046B. A vector, referred to as gradient vector gradᵢ, is determined by computing the average of the first VA and second VB computing values weighted by the relative amplitude amp. In other words, the gradient vector gradᵢ complies with the following equation:

$${grad}_{i} = \frac{VA - VB}{2 * amp} \qquad [ MATH 4 ]$$

where gradᵢ is the gradient associated with the i-th ingredient 2014 of the initial cosmetic composition 2011i.

The computations of pairs of computing cosmetic compositions 2045A, 2045B, 2046A, 2046B, of computing values VA, VB, and of gradient vector gradᵢ, are repeated for each ingredient 2014 of the initial cosmetic composition 2011ᵢ, such that a respective gradient vector gradᵢ of each ingredient 2014 is obtained.

These gradient vectors gradᵢ are then arranged in a matrix forming the Jacobian matrix J, for example according to the following equation:

$$J = [ g r a {d}_{1} … {grad}_{i} … {grad}_{n} ] \qquad [ MATH 5 ]$$

where n is the number of ingredients 2014 in the initial cosmetic composition 2011ᵢ.

In addition, for any cosmetic composition 2011 of which the quantity of each ingredient 2014 differs from the initial cosmetic composition 2011ᵢ by a magnitude Δmpᵢ, a value V of the at least one associated colorimetric parameter is expressed, by first order linearization of the automatic determination model, with the following equation:

$$V = Vi + J Δ mp \qquad [ MATH 6 ]$$

where Vi is the initial value,
Δmp is a vector comprising for each ingredient, the magnitude Δmpᵢ of variation of the quantity of said ingredient.

The processing phase 2200 further comprises a step 2250 of adapting the initial cosmetic composition 2011ᵢ for compensate for the first deviation between the initial value Vi and the target value Vc.

The adaptation step 2250 comprises varying the quantity of each ingredient 2014 in the initial cosmetic composition 2011ᵢ to form for example a varied cosmetic composition of which the quantities of each ingredient comply with the following equation:

$$m {p}_{ν} = m p + Δ m p \qquad [ MATH 7 ]$$

where mpᵥ is a vector comprising the quantity of each ingredient 2014 of the varied cosmetic composition,
mp is a vector comprising the quantity of each ingredient of the initial cosmetic composition 2011ᵢ, and
Δmp is a vector comprising, for each ingredient 2014, the variation of the quantity of this ingredient 2014.

The adaptation step 2250 then comprises evaluating, using the automatic determination model, the influence of each variation on the value of the at least one colorimetric parameter.

Preferably, this influence is evaluated by computing the value of a cost function FC, optionally dependent on the initial value Vi, the target value Vc, the Jacobian matrix J and said variation.

Preferably, the cost function FC complies with the following equation:

$$FC = ‖ Vi - V c + J Δ mp ‖ \qquad [ MATH 8 ]$$

where ∥·∥ is a mathematical norm.

It is then understood that if the value of the cost function is equal to zero, then the first deviation is compensated. However, this value of the cost function FC is not always attainable.

Preferably, during the adaptation step 2250, the variation and the evaluation are repeated several time by an optimization algorithm aimed at minimizing the cost function value FC. At each repetition, the optimization algorithm determines the variation of the quantity of each ingredient 2014 according to the cost function value FC of the preceding repetition. Preferably, the variations are determined using the Jacobian matrix J. Indeed, the initial Vi and target Vc values are constants for the optimization algorithm.

The problem solved by the optimization algorithm can then be formulated thus:

$${min}_{Δ m p} F C \qquad [ MATH 9 ]$$

According to a variant, the optimization algorithm applied is an optimization algorithm constrained by the set E_{cv} of cosmetic composition viability constraint(s). The optimization problem solved by the optimization algorithm can then be formulated as follows:

$${min}_{Δ m p} ( FC ) \qquad [ MATH 10 ]$$

$$such that mp + Δ mp ∈ {E}_{c ν}$$

where E_{cv} is the set of viability constraints.

According to a variant, the cost function FC is penalized by a magnitude representative of the variation of the quantity of each ingredient 2014 and dependent on a predefined coefficient α. In other words, the cost function FC complies for example with the following equation:

$$FC = ‖ Vi - Vc + J Δ mp ‖ ++ α {‖ Δ mp ‖}^{2} \qquad [ MATH 11 ]$$

Penalizing the cost function FC ensures that the cosmetic composition obtained for the following repetition is not too different from the initial composition 2011ᵢ. The preceding considerations on the optimization problems of equations 9 and 2010 also apply when the cost function FC complies with equation 11 rather than equation 8.

The solution of the optimization problem supplies the optimized variations Amp* of the quantity of each ingredient 2014 of the initial cosmetic composition 2011ᵢ.

Following the adaptation step 2250, an adapted cosmetic composition 2011* is obtained. The adapted cosmetic composition 2011* comprises the same ingredients 2014 as the initial cosmetic composition 2011ᵢ. The quantity of each ingredient 2014 of the adapted cosmetic composition 2011* differs from the quantity of said ingredient 2014 in the initial composition 2011ᵢ, by the corresponding optimized variation.

When the optimization problem solved by the optimization algorithm is the problem of the equation 2010, the initial cosmetic composition 2011ᵢ and the adapted cosmetic composition 2011* comply with the set E_{cv} of viability constraint(s).

Furthermore and optionally, the processing phase 2200 further comprises a step 260 of sending the adapted cosmetic composition 2011*, to the display screen 2016. The display screen 2016 then displays the adapted cosmetic composition 2011*, intended for the operator.

According to another optional addition, the processing phase 2200 further comprises a step 2270 of producing at least one sample of adapted cosmetic composition 2011*. The at least one sample is preferably produced by the unit 2017 for producing cosmetic compositions.

The sample of the adapted cosmetic composition 2011ᵢ is intended to be applied on hair 2012, preferably a lock of hair 2012, to verify the value of the at least one colorimetric parameter.

Thus, following the application of the sample on hair 2012, an adapted value Va of the at least one colorimetric parameter of the adapted cosmetic composition 2011* is measured using the same sensors used to form the training data 2018.

The adapted cosmetic composition 2011* being such that a second deviation between the adapted value Va and the target value Vc is less than the first deviation.

Preferably, the processing phase 2200 is repeated at least once forming at least two iterations. At each repetition, during the step 2220 of obtaining the initial cosmetic composition 2011ᵢ, said initial cosmetic composition 11; is the adapted composition 2011* of the preceding processing phase 2200. Thus, none of the sub-steps 2221, 2222, 2223, 2224, 2225, 2226, 2227, 2228 are implemented again.

At each repetition, the step 2210 of obtaining the target value is preferably not implemented.

At each repetition, during the step 2230 of obtaining the initial value Vi of the at least one colorimetric parameter, said initial value Vi is the adapted value Va of the preceding processing phase 2200. At each repetition, during the optional step 2240 of computing the Jacobian matrix J, the operating point for which the Jacobian matrix J is computed is the adapted cosmetic composition 2011* of the preceding processing phase 2200.

Thus, for each repetition of the processing phase 2200, an adapted value Va specific to the repetition is measured. Preferably, the adapted values form a sequence converging toward the target value Vc.

In addition, following the repetition(s) of the processing phase 2200, an adapted cosmetic composition 2011* obtained has an adapted value Va of the at least one colorimetric parameter, equal to the target value Vc.

Alternatively, each cosmetic composition is capable of being applied to any type of hairs 2012, and not only hair, for example to eyelashes, eyebrows or a beard of the user.

The variants and addition described above can be combined in any technical possible combination.

1. A method for determining at least one colorimetric parameter characterizing a cosmetic composition for coloring hairs, the method being implemented by an electronic determination device and comprising a training phase including the following steps:
receiving a set of viability constraint(s) of cosmetic composition(s),
acquiring a set of training data, each training data item being specific to a cosmetic composition complying with the set of viability constraint(s) received, each training data item comprising:
a set of magnitudes representing the quantity of each ingredient of the cosmetic composition, and
a value of the at least one colorimetric parameter associated with the cosmetic composition, and
training an artificial intelligence model based on the set of training data, to obtain a trained model,
the method further comprising a processing phase including the following steps:
obtaining a test data item relating to a cosmetic composition under test, the cosmetic composition under test complying with the set of viability constraint(s) received during the training phase,
the test data item comprising a set of magnitudes representing the quantity of each ingredient of the cosmetic composition under test,
applying, to the test data item obtained, the trained model to determine the value of the at least one colorimetric parameter of the cosmetic composition under test.

2. The method according to claim 1, wherein each ingredient of the test data item relating to the cosmetic composition under test is chosen from a predefined list of ingredients.

3. The method according to claim 2, wherein during the obtaining step of the processing phase, several test data items are obtained, each test data item being specific to a respective cosmetic composition under test, during the application step of the processing phase, the trained model is applied to each test data item obtained to determine a value of the at least one colorimetric parameter associated with said test data item,
the obtaining step of the processing phase comprising:
receiving a number N corresponding to a number of cosmetic compositions under test, the number N being greater than or equal to two,
generating N test data items,
each test data item relating to a cosmetic composition of which the ingredients are chosen from the predefined list of ingredients and such that the cosmetic composition associated with each test data item is within a reduced viable cosmetic composition space,
the reduced viable cosmetic composition space only comprising cosmetic compositions complying with the set of viability constraint(s), and
the N test data items being representative of said reduced viable cosmetic composition space.

4. The method according to claim 3, wherein, during the obtaining step of the processing phase, generating N test data items of the obtaining step comprises computing magnitudes of the N test data items such that, for each test data item, the distance between said test data item and the other test data items is maximum,
each magnitude of each test data item representing the quantity of an ingredient in the corresponding cosmetic composition under test.

5. The method according to claim 2, wherein the predefined list of ingredients comprises one or more ingredients of a first type, and one or more ingredients of a second type,
the or each ingredient of the first type being a base, and
the or each ingredient of the second type being a coupler.

6. The method according to claim 5, wherein the set of cosmetic composition viability constraint(s) comprises one or more of the following constraints:
a ratio between a quantity of ingredient of the first type and a quantity of ingredient of the second type is between a first threshold and a second threshold,
a total quantity of the cosmetic composition is less than a third threshold,
a quantity of each ingredient in the cosmetic composition is less than a fourth threshold,
a number of ingredients in the cosmetic composition is less than a fifth threshold.

7. The method according to claim 1, wherein the at least one colorimetric parameter comprises:
a triplet of values characterizing a hair color after applying the cosmetic composition, or
a color fade value of the cosmetic composition after washing the hairs, or
a selectivity value characterizing a difference in color between a root and a tip of the hairs.

8. The method according to claim 1, wherein during the acquisition step of the training phase, the set of training data is, at least in part, acquired via a sensor capable of measuring the at least one colorimetric parameter.

9. The method according to claim 1, wherein the processing phase further comprises a step of sending the or each colorimetric parameter and the associated test data item, to a display screen, with a view to displaying on the display screen, a rendering for the or each cosmetic composition under test, the or each rendering being representative of the application, to the hairs, of the cosmetic composition under test.

10. The method according to claim 1, wherein the processing phase further comprises a step of producing at least one sample of cosmetic composition under test, with a view to applying it on hairs to verify the value of the at least one colorimetric parameter.

11. A computer program product on which a computer program comprising program instructions is stored, the computer program being loaded onto a data processing unit and implementing a method according to claim 1 when the computer program is implemented on the data processing unit.

12. An electronic device for determining at least one colorimetric parameter characterizing a cosmetic composition for coloring hairs,
the electronic determination device being capable of implementing a determination method according to claim 1.

13. A method for determining a target cosmetic composition for coloring hairs, in particular hair, according to at least one target colorimetric parameter,
the method being implemented by an electronic determination device and comprising a processing phase comprising the following steps:
acquiring the at least one target colorimetric parameter,
obtaining a set of cosmetic compositions under test, each cosmetic composition under test comprising ingredients,
obtaining at least one colorimetric parameter associated with each cosmetic composition under test,
filtering the cosmetic compositions under test according to the at least one colorimetric parameter associated with them, to obtain filtered cosmetic compositions, the at least one colorimetric parameter associated with each filtered cosmetic composition meeting a criterion relating to the at least one target colorimetric parameter,
forming several clusters of filtered cosmetic compositions such that the filtered cosmetic compositions from the same cluster comprise the same ingredients,
selecting a cluster of filtered cosmetic compositions, referred to as selected cluster, and
determining the target cosmetic composition according to the filtered cosmetic compositions of the selected cluster.

14. The method according to claim 13, wherein each filtered cosmetic composition comprises, for each of its ingredients, a quantity of said ingredient in the filtered cosmetic composition,
the determination step of the processing phase comprising:
for each ingredient of the filtered cosmetic compositions of the selected cluster, computing an average value of the quantities of said ingredient from said filtered cosmetic compositions,
determining an average cosmetic composition comprising, for each of said ingredients, a quantity equal to the respective computed average value of the ingredient, and
forming the target cosmetic composition according to the average cosmetic composition.

15. The method according to claim 13, wherein during the determination step of the processing phase, forming the target cosmetic composition comprises computing at least one colorimetric parameter associated with the average cosmetic composition and optimizing the quantity of each ingredient of the average cosmetic composition such that the at least one colorimetric parameter associated with the average cosmetic composition approaches the at least one target colorimetric parameter,
the target cosmetic composition being the average cosmetic composition resulting from optimizing its quantities of ingredients.

16. The method according to claim 1, wherein during the selection step of the processing phase, M clusters of filtered cosmetic compositions are selected, M being greater than or equal to two, during the determination step of the processing phase, for each selected cluster, a corresponding target cosmetic composition being determined.

17. The method according to claim 16, wherein during the selection step of the processing phase, the M clusters are selected such that, for each selected cluster, the ingredients comprised in the filtered cosmetic compositions of said selected cluster are only comprised in said selected cluster.

18. The method according to claim 13, wherein the method comprises, prior to the processing phase, a training phase including the following steps:
receiving a set of viability constraint(s) of cosmetic composition(s),
acquiring a set of training data, each training data item being specific to a cosmetic composition complying with the set of viability constraint(s) received,
each training data item comprising:
a set of magnitudes representing the quantity of each ingredient of the cosmetic composition, and
a value of the at least one colorimetric parameter associated with the cosmetic composition, and
training an artificial intelligence model based on the set of training data, to obtain a trained model,
during the step of obtaining at least one colorimetric parameter associated with each cosmetic composition under test, of the processing phase, each colorimetric parameter being obtained by applying the trained model to a corresponding cosmetic composition under test.

19. The method according to claim 13, wherein the step of obtaining a set of cosmetic compositions under test, of the processing phase comprises:
receiving a number N corresponding to the number of cosmetic compositions under test, the number N being greater than or equal to two,
generating N test data items,
each test data item relating to a cosmetic composition of which the ingredients are chosen from a predefined list of ingredients and such that the cosmetic composition associated with each test data item is within a reduced viable cosmetic composition space, the reduced space only comprising the cosmetic compositions complying with a set of viability constraint(s), and
the N test data items being representative of said reduced viable cosmetic composition space,
the set of cosmetic compositions under test being formed from the N test data items generated.

20. The method according to claim 19, wherein, during the processing phase, generating N test data items of the obtaining step comprises computing magnitudes of the N test data items such that, for each test data item, the distance between said test data item and the other test data items is maximum,
each magnitude of each test data item representing the quantity of an ingredient in the corresponding cosmetic composition under test.

21. The method according to claim 13, wherein the at least one colorimetric parameter comprises:
a triplet of values characterizing a hair color after applying the cosmetic composition, or
a color fade value of the cosmetic composition after washing the hairs, or
a selectivity value characterizing a difference in color between a root and a tip of the hairs.

22. The method according to claim 13, wherein the processing phase further comprises a step of producing at least one sample of the target cosmetic composition, with a view to applying it on hairs to verify the value of the at least one colorimetric parameter.

23. A computer program product on which a computer program comprising program instructions is stored, the computer program being loaded onto a data processing unit and implementing a method according to claim 13 when the computer program is implemented on the data processing unit.

24. An electronic device for determining a target cosmetic composition for coloring hairs, in particular hair, according to at least one target colorimetric parameter, the electronic determination device comprising a processing unit capable of implementing a method according to claim 13.

25. A method for adapting an initial cosmetic composition, intended for coloring hairs, in particular hair, so that the value (Va) of at least one colorimetric parameter of the adapted cosmetic composition corresponds to a target value (Vc),
the method being implemented by an electronic adaptation device and comprising a processing phase including the following steps:
obtaining a target value (Vc) for the at least one colorimetric parameter,
obtaining data relating to an initial cosmetic composition including a plurality of ingredients and for each ingredient, an associated quantity,
obtaining an initial value (Vi) of the at least one colorimetric parameter for the initial cosmetic composition, the initial value (Vi) resulting from a measurement following the application of the initial cosmetic composition on hairs, the initial value (Vi) differing from the target value (Vc) by a first deviation,
adapting the initial cosmetic composition to compensate for the first deviation between the initial value (Vi) and the target value (Vc), the adaptation comprising:
varying the quantity of each ingredient in the initial cosmetic composition, and
evaluating, using an automatic determination model, the influence of each variation on the value of the at least one colorimetric parameter, the automatic determination model having been previously trained to determine a value of at least one colorimetric parameter according to a cosmetic composition,
the adapted cosmetic composition being obtained following the adaptation.

26. The method according to claim 25, wherein the adapted cosmetic composition has an adapted value (Va) for the at least one colorimetric parameter, the adapted value (Va) resulting from a measurement following the application of the adapted cosmetic composition on hairs, the adapted cosmetic composition being such that a second deviation between the adapted value (Va) and the target value (Vc) is less than the first deviation.

27. The method according to claim 25, wherein the processing step further comprises the following step:
computing, using the automatic determination model, a matrix, referred to as Jacobian matrix (J), representing a variation of the value of the at least one colorimetric parameter of the initial cosmetic composition according to a variation of the quantity of each ingredient of the initial cosmetic composition,
during the evaluation of the influence of each variation on the value of the at least one colorimetric parameter of the adaptation step (2250) of the processing phase, said influence being evaluated by computing a value of a cost function (FC) dependent on the Jacobian matrix (J) and each variation.

28. The method according to claim 27, wherein the cost function (FC) complies with the following formula:
$$FC = ‖ Vi - Vc + J Δ mp ‖$$
where FC is the cost function,
Vi is the initial value of the at least one colorimetric parameter,
Vc is the target value,
J is the Jacobian matrix, and
Δmp is a vector comprising each ingredient quantity variation.

29. The method according to claim 25, wherein during the adaptation step of the processing phase, the variation and the evaluation are repeated several times by an optimization algorithm.

30. The method according to claim 29, wherein during the adaptation step of the processing phase, the optimization algorithm applied is an optimization algorithm constrained by a set (Ecv) of viability constraint(s) of the cosmetic composition(s),
the initial cosmetic composition and the adapted cosmetic composition complying with the set (Ecv) of viability constraint(s).

31. The method according to claim 25, wherein the method comprises, prior to the processing phase, a training phase including the following steps:
receiving a set of viability constraint(s) of cosmetic composition(s),
acquiring a set of training data, each training data item being specific to a cosmetic composition complying with the set of viability constraint(s) received,
each training data item comprising:
a set of magnitudes representing the quantity of each ingredient of the cosmetic composition, and
a value of the at least one colorimetric parameter associated with the cosmetic composition, and
training an artificial intelligence model based on the set of training data, to obtain a trained model capable of determining, from a cosmetic composition, a value of the at least one colorimetric parameter of the cosmetic composition,
during the adaptation step of the processing phase, the model for determining at least one cosmetic composition colorimetric parameter being the model trained during the training step of the training phase.

32. The method according to claim 25, wherein the processing phase further comprises a step of producing a sample of the adapted cosmetic composition, in order to measure the adapted value (Va) of the at least one colorimetric parameter of the adapted colorimetric composition.

33. The method according to claim 25, wherein the processing phase is repeated at least once forming at least two iterations,
during the steps of obtaining the or each repetition of the processing phase, the initial cosmetic composition being the adapted cosmetic composition obtained in the preceding iteration and the initial value (Vi) resulting from a measurement following the application of the adapted cosmetic composition from the preceding iteration on hairs.

34. A computer program product on which a computer program comprising program instructions is stored, the computer program being loaded onto a data processing unit and implementing a method according to claim 25 when the computer program is implemented on the data processing unit.

35. An electronic device for adapting an initial cosmetic composition, intended for coloring hairs, so that the value of at least one colorimetric parameter of the adapted cosmetic composition corresponds to a target value, the electronic adaptation device being capable of implementing a determination method according to claim 25.
